(19) United States
(12) Patent Application Publication
(10) Pub. No.: US 2022/0265175 A1
(43) Pub. Date: Aug. 25, 2022

(54) SYSTEMS, METHODS AND DEVICES FOR PREDICTING AND DETECTING POSTOPERATIVE COMPLICATIONS

(71) Applicant: NERV TECHNOLOGY INC., Kitchener (CA)

(72) Inventors: Youssef HELWA, Waterloo (CA); Abdallah EL-FALOU, Mississauga (CA); Mohamed OKASHA, Waterloo (CA); Amr ABDELGAWAD, Kitchener (CA); Kenan HABIB, Milton (CA); Khaled BERRY, Oakville (CA); Ricky TJANDRA, Waterloo (CA)

(21) Appl. No.: 17/598,118

(22) PCT Filed: Mar. 26, 2020
(86) PCT No.: PCT/CA2020/050395

§ 371 Date: Sep. 24, 2021

Related U.S. Application Data
(60) Provisional application No. 62/823,897, filed on Mar. 26, 2019.

(51) Int. Cl. A61B 5/145; A61B 5/00; A61B 5/01; A61B 5/1473

(52) U.S. Cl. CPC A61B 5/14507; A61B 5/4255; A61B 5/6852; A61B 5/01; A61B 5/14539; A61B 5/14546; A61B 5/1473; A61B 2505/05; A61B 2505/07

(57) ABSTRACT

A monitoring device includes an input port attachable for fluid communication with a catheter, the catheter for insertion in a body of a user, for receiving fluid from the body of the user, an output port, generally parallel to the input port, in fluid communication with a fluid reservoir, a fluid channel defining fluid communication between the input port and the output port, and a biosensor for continuously measuring bio-signal data of the fluid in the fluid channel, the biosensor including an electrode pair. The biosensor is in communication with a computing device for determining a condition of the user based at least in part on the bio-signal data.

### CROSS-REFERENCE TO RELATED APPLICATIONS

This application claims priority from U.S. Provisional Patent Application No. 62/823,897 filed on Mar. 26, 2019, the contents of which are hereby incorporated by reference.

### FIELD

The present disclosure relates to systems, methods and devices for monitoring, predicting and detecting different forms of post-operative complications.

### BACKGROUND

Surgical procedures may use open and minimally invasive techniques on users, such as patients, in order to identify and treat pathological conditions or improve body functions. Surgeries performed due to a variety of reasons have an inherent risk of post-operative complications such as hemorrhages, infections and leakages to develop.

One of the most dangerous complications for surgery is a complication known as anastomotic leakage. Anastomotic leakage may develop after an anastomosis is performed where two organs are surgically connected, and is most commonly observed in gastrointestinal surgery. Anastomotic leakage leads to luminal contents leaking into the peritoneal cavity which may cause a cascade of deadly complications to arise. This typically involves a form of severe sepsis, peritonitis, morbidity and it may lead to mortality.

Using traditional techniques, it can take three to seven days on average for a leak to be diagnosed. This is very dangerous especially considering that every hour of delay causes a considerable increase in the morbidity and mortality risk for the patient.

Typically, medical facilities wait for clinical factors such as abdominal pain, fever and tachycardia to arise before diagnosis. Existing technologies for detection of post-operative complications, such as anastomotic leakage, may be nonspecific, inefficient, time-consuming, expensive, and/or lacking in the ability to provide real-time detection of the complication.

### SUMMARY

Leak incidence rate from surgical procedures can vary from 1% to 40% in some cases. Causes behind the development of anastomotic leaks are still being studied with no definitive causes identified yet. There are however risk factors that are associated with higher incidence rate such as age, gender, organ tension, local ischemia, medical history, and surgical errors.

Physiological changes may occur before and during the leakage development, including tissue necrosis which is most typically seen at the surgical site and also caused by local ischemia. Another change that may be seen is decay of the surgical site, which may be associated with the failure of the staple line.

Leakages that may develop across different organs include intraperitoneal gastric, fecal, urinary and bile leaks, which are typically difficult to diagnose early. Other forms of luminal leaks included leakage of saliva or gastric contents in thoracic surgeries. In addition to the different forms of luminal leaks that could arise, different forms of hemorrhages could arise after surgeries.

Imaging techniques such as Computed Tomography (CT) imaging can be used for leakage detection, however, CT imaging has certain drawbacks, especially due to its low sensitivity, ionizing radiation and the long time it takes to acquire and assess an image. Such systems and technologies require hospital facilities and trained personnel from different specialty teams in order to operate these technologies. Additionally, hospital facilities have to be equipped with high standard equipment and timed reporting systems in order to ensure proper detection of these postoperative complications within a timely manner.

Other techniques which may decrease the probability of a leak developing during surgery include imaging techniques designed to monitor the blood supply to the region. Such imaging techniques may decrease the risk of leaks in some cases but may not prevent future leaks from happening.

Other methods for detecting post-operative complications, such as leakage complications, may include gas sensors (that monitor certain analytes such as CH₄ and N₂). However, most of these techniques suffer from major setbacks associated with sensitivity, specificity, cost, and applicability in the clinical setting.

The present disclosure provides systems, methods and devices for analyzing bodily and luminal fluids, which includes, without limitation, peritoneal fluid, peritoneal drainage fluid, pleural drainage fluid, gastric juice, fecal matter, bile fluid, urine, amniotic fluid, dialysate, sebum, or blood. The fluid may be continuously monitored for changes and trends in specific analytes and biological properties. Examples of these properties and analytes include, but are not limited to, pH, lactate, electrolytes, impedance, conductivity, dissolved oxygen, dissolved CO2, temperature, inflammatory markers, enzymes, bacterial proteins, RNA or lipids. Systems, methods and devices disclosed herein may be used for various diagnostic applications such as, but not limited to, post-operative leakages, ischemia, infection, and sepsis.

In some embodiments, sensors such as biosensors may be placed on a catheter, and the catheter may be inserted into the body and may allow fluid to be injected into or withdrawn from the body. The catheter can be placed proximal to the surgical site in order to monitor the milieu of the biological fluid proximate to the region. The fluid can be directly sensed locally without the need for negative pressure or it can use negative pressure to assist the fluid to be driven through the catheter. Any number of sensors can be placed on the surface of the catheters such that they are directly in contact with the biological fluid surrounding the area of interest such as the suture line in the case of an anastomosis for example. Sensors may also be placed on the inside of the catheter, a balloon, a pump or any tubing where the fluid can be collected.

In a further embodiment, sensors may be housed within a system that can be placed inline with a catheter. The catheter can be placed proximal to the surgical site in order to monitor the milieu of the peritoneal fluid proximate to the region. The system may be an extension of an existing catheter system. The system may be attached at any time, when the catheter is being placed or at a later date.

Conveniently, early management to handle complications and address leakages, using techniques disclosed herein, may significantly mitigate risks associated with such complications.

Existing techniques of handling of complications may involve using interventional radiology techniques to handle existing complications with patients. In the case of leakages this may involve techniques such as placing in drains, placing stents, enforcing the staple line sutures. These interventions may be done endoscopically without the need for a second surgery to be performed. Monitoring the user status using systems and techniques as disclosed herein may allow for a more effective treatment plan and earlier intervention if the complication appears again.

In addition, techniques disclosed herein may allow for home monitoring of the post-operative journey as more patients are moved to out patient monitoring settings. Furthermore, techniques disclosed herein may allow for users to continuously monitor the status of a patient-surgery, which may be an improvement over existing diagnostic tests that take a sample at a specific point in time, which may not be indicative of a patient's status.

According to an aspect, there is provided a monitoring device comprising: an input port attachable for fluid communication with a catheter, the catheter for insertion in a body of a user, for receiving fluid from the body of the user; an output port, generally parallel to the input port, in fluid communication with a fluid reservoir; a fluid channel defining fluid communication between the input port and the output port; and a biosensor, in communication with a computing device, for continuously measuring bio-signal data of the fluid in the fluid channel, the biosensor including an electrode pair.

In some embodiments, the computing device is for determining a condition of the user based at least in part on the bio-signal data.

In some embodiments, the biosensor includes an impedance sensor for detecting a conductivity of the fluid in the fluid channel.

In some embodiments, the biosensor includes a pH sensor for detecting a pH level in the fluid in the fluid channel.

In some embodiments, the biosensor includes at least one of a lactate sensor, an amylase sensor, a urea sensor, or a creatinine sensor.

In some embodiments, the device further comprises a flow sensor for continuously determining a flow rate of the fluid in the fluid channel over time.

In some embodiments, the device further comprises a light-based sensor including a light transmitter and a light receiver for detecting transmission of light through the fluid in the fluid channel.

In some embodiments, the light-based sensor is configured to detect a colour of the fluid based at least in part on a detected wavelength.

In some embodiments, the device further comprises a temperature sensor for detecting a temperature of the fluid in the fluid channel.

In some embodiments, the biosensor is disposed on a substrate in fluid communication with the fluid channel.

In some embodiments, the electrode pair is disposed sequentially along a length of the fluid channel.

According to another aspect, there is provided a computer-implemented method for monitoring a user, the method comprising: receiving bio-signal data continuously from a biosensor in fluid communication with the fluid; determining a condition of the user based at least in part on the bio-signal data; and predicting a future occurrence of a complication based at least in part on the condition of the user.

In some embodiments, the method further comprises receiving a profile of the user, the profile of the user including information related to a surgical procedure performed on the user, wherein the future occurrence of the complication is predicted based at least in part on the profile of the user.

In some embodiments, the method further comprises updating the profile of the user based at least in part on the bio-signal data.

In some embodiments, the method further comprises receiving flow data continuously from a flow sensor in fluid communication with fluid from a body of a user; and determining, based at least in part on the flow data, a rate of flow of the fluid, wherein the condition of the user is determined based at least in part on the rate of flow.

In some embodiments, the method further comprises determining a change in the rate of flow of the fluid over time and a change in bio-signal data over time, and the predicting the future occurrence is based at least in part on the change in the rate of flow and the change in bio-signal data.

In some embodiments, the flow data is received in near real-time.

In some embodiments, the bio-signal data is received in near real-time.

In some embodiments, the method further comprises receiving light data associated with transmission of light through the fluid from a light-based sensor in fluid communication with the fluid.

In some embodiments, the method further comprises determining a color of the fluid based at least in part on the light data.

In some embodiments, the method further comprises receiving temperature data of the fluid from a temperature sensor in fluid communication with the fluid.

In some embodiments, the method further comprises modulating the bio-signal data based at least in part on the temperature data.

In some embodiments, the method further comprises determining a risk factor of the user based on a cross-correlation with a trend of bio-signal data of other users.

In some embodiments, the condition of the user is based at least in part on determining whether the bio-signal data is within bounds of a threshold.

According to a further aspect, there is provided a system for monitoring a user, comprising: a processor; a memory in communication with the processor, the memory storing instructions that, when executed by the processor cause the processor to perform a method as described herein.

Other features will become apparent from the drawings in conjunction with the following description.

### BRIEF DESCRIPTION OF DRAWINGS

In the figures which illustrate example embodiments:

FIG. 1 is a diagram of a system utilized to detect a surgical leak, including a sensor device having a catheter embedded with biosensors, proximate to a surgical site and with data visualized on a remote device, in accordance with an embodiment.

FIGS. 2A, 2B and 2C illustrate an enlarged view of different types of biosensors integrated onto a catheter of a sensor device, in accordance with embodiments.

FIGS. 3A, 3B and 3C illustrate an enlarged view of different types of biosensors integrated inside catheter lumens of a sensor device, in accordance with embodiments.

FIGS. 4A, 4B, 4C and 4D illustrate an enlarged view of catheter lumens and different configurations for fluids and wires of a sensor device, in accordance with embodiments.

FIGS. 5A, 5B, 5C illustrate different configurations of a system including a sensor device, placed in an abdomen for diagnostic applications of Gastrointestinal (GI) surgery, in accordance with embodiments.

FIG. 5D is a schematic diagram of a system including a sensor device, in accordance with an embodiment.

FIG. 5E illustrates a configuration of the system of FIG. 5D, with the sensor device placed in a pleural cavity adjacent a thorax, in accordance with an embodiment.

FIG. 5F illustrates a system including multiple sensor devices, in accordance with an embodiment.

FIG. 5G is a front view of the sensor device of FIG. 5D.

FIG. 5H is a side perspective view of the sensor device of FIG. 5D.

FIG. 5I is a top perspective view of the sensor device of FIG. 5D.

FIGS. 6A, 6B illustrate an enlarged view of biosensors of a sensor device that can be connected wirelessly to an external receiver, in accordance with an embodiment.

FIG. 7 illustrates an example system design that may be utilized to collect signals from the biosensors and relay them to the users.

FIG. 8 is a process flowchart showing a system for detecting an anastomotic leak, in accordance with an embodiment.

FIG. 9 is a graph illustrating a form of a readout that may be obtained from a data acquisition system, in accordance with an embodiment.

FIG. 10 is a block diagram of example hardware components of a computing device of the system of FIG. 1, in accordance with an embodiment.

FIG. 11 illustrates the organization of software at the computing device of FIG. 10, in accordance with an embodiment.

FIG. 12A is a flow chart of a method for detecting or predicting a leak, performed by the software of FIG. 11, in accordance with an embodiment.

FIG. 12B is a flow chart of a method for monitoring a user, performed by the software of FIG. 11, in accordance with an embodiment.

FIG. 13 illustrates a table of reported GI tract pH values in a human.

FIG. 15A illustrates sensor data captured during a study.

FIG. 15B illustrates details of the study of FIG. 15A.

FIG. 16A is a perspective view of a sensor assembly of an inline monitoring device, in accordance with an embodiment.

FIG. 16B is a cross-section view of the sensor assembly of FIG. 16A along lines I-I.

FIG. 16C is a cross-section view of the sensor assembly of FIG. 16A along lines I-I and having a suspended particle in a fluid channel.

FIG. 17A is a perspective view of a sensor assembly including a light-based sensor disposed on two substrates, in accordance with an embodiment.

FIG. 17B is a cross-section view of the sensor assembly of FIG. 17A along lines II-II.

FIG. 18A is a perspective view of a sensor assembly having a fibre optic light-based sensor, in accordance with an embodiment.

FIG. 18B is a cross-section view of the sensor assembly for FIG. 18A along lines III-III.

FIG. 19A is a perspective view of a sensor assembly having a light-based sensor, in accordance with an embodiment.

FIG. 19B is a cross-section view of the sensor assembly of FIG. 19A along lines IV-IV.

### DETAILED DESCRIPTION

Systems, methods and devices disclosed herein can be utilized for monitoring, detecting and predicting different forms of postoperative complications, such as leakage, that can arise following surgeries. Embodiments can include a sensing and diagnostic device that utilizes sensors, for example, on a catheter or an inline device, to detect or predict, for example, the presence of luminal fluid when a leak develops.

In some embodiments, systems, methods and devices disclosed herein include sensors, such as biosensors that can be used to sense bio-signal data, placed at locations proximate to the surgical site, enabling the monitoring of biological fluids for analytes that could be indicative of a surgical leak.

In some embodiments, sensors may include electrochemical or solid-state sensors with different forms, which include but are not limited to potentiometric, voltammetric, conductometric, capacitive, amperometric or ion-sensitive field effect transistors (ISFET). In some embodiments, sensors may be piezoelectric or micro-electro-mechanical systems (MEMS). Sensors may include terminals that connect to active, counter, reference or pseudo-reference electrodes depending on the type of sensor being utilized. Sensors can be of different types that include but are not limited to pH sensors, ion-sensitive sensors, temperature sensors, lactate sensors, electrolyte sensors, impedance sensors, fluid sensors, light-based sensors, microorganism sensors, protein sensors, inflammatory sensors, carbohydrate sensors, enzyme sensors, oxygen sensors such as PO2 (partial pressure of oxygen) sensors, amylase sensors, urea sensors, creatinine sensors, pressure sensors and flow sensors.

Sensors may be connected in series or in parallel, and may be disposed disposed sequentially, for example, along a length of a fluid channel.

In some embodiments, sensors may include a temperature sensor, such as a thermistor.

In use, a thermistor may undergo changes in resistance correlated to changed in temperature. Thus, a temperature may be determined by determining a resistance of the thermistor, by exciting with current and measuring voltage (or vice versa).

A temperature sensor may be used to account for a number of artifacts and error sources in the biosignal measurements. A temperature sensor may be used to compensate or modulate signals from other sensors that are temperature dependent such as impedance and pH. A rise in fluid temperature detected by temperature sensor can indicated an influx of new fluid, as biological fluids tend to have higher temperatures relative to ambient temperatures.

An array of temperature sensors and a heating element may be used to measure fluid flow rate using the principles of thermal mass fluid transport.

In some embodiments, sensors may include a flow sensor such as a flowmeter to measure the volumetric or mass flow rate of a fluid such as a liquid or a gas, for example, in a user's body.

In some embodiments, sensors may include a pH sensor that is electrochemical in nature allowing biological analytes to be transduced into electrical signals that can be then measured, monitored and analyzed to determine if a postoperative complication is developing. A system of interdigitated electrodes (active, counter and reference) may be fabricated on a biocompatible substrate. The electrodes may be fabricated from biocompatible materials: gold, platinum, titanium and silver, and then later functionalized with an active polyaniline (PANI) polyaniline/polyurethane (PAIN/PU), polyurethane, polymer or other suitable layer. In an example, p-biosensors are 500 μm×500 μm in size, allowing them to be placed on catheters to monitor changes in pH over time.

A pH sensor may be formed from a conducting polymer made from Aniline monomers. A sensitivity to pH levels of a suitable conducting polymer can allow for its use as a pH sensitive component in pH sensors.

A pH sensor may be calibrated and/or controlled by a potentiostat, in particular, an electronic device that controls the difference in potential and current of a 3-electrode system comprising of a working electrode (WE), a reference electrode (RE) as well as a counter electrode (CE). This electrical instrument has many applications that may be used to fabricate a pH sensor such as Cyclic Voltammetry (CV), Chronoamperometry and Chronopotentiometry.

A pH sensor may be configured to detect a pH value within a threshold or boundaries, or deviation from such boundaries.

In some embodiments, sensors may include a light-based sensor, such as photoelectric sensors, utilizing a combination of light transmitters or sources and detectors in the ultraviolet to infrared spectrum to measure the fluid's light absorption or transmission characteristics. Single-wavelength or multi-wavelength rays may be used.

Light-based sensors can include a combination of light transmitters and detectors in the ultraviolet to infrared spectrum and be used to measure a fluid's light absorption or transmission characteristics.

Light absorption or transmission characteristics can be indicative of changes in the bodily and luminal fluids that can include, but are not limited to, protein composition and concentration, pH, conductivity, inflammatory markers and cellular activities due to onset of complications or disease. This also enables measurement of the fluid's color, which can be indicative of bleeding (red), bile leaks (green-yellow), fecal leaks (brown), gastric leaks (green), urine leaks (yellow), and other fluids of specific colors.

In some embodiments, single-wavelength or multi-wavelength rays may be used. Changes detected in the absorption or transmission characteristics of fluids within specific light bands or wavelength may enable measurement of a fluid's color. Since serous fluids (for example, peritoneal and pleural fluids) are typically pale yellow, a change in color may be indicative of bleeding (red), bile leaks (green-yellow), fecal leaks (brown), gastric leaks (green), urine leaks (yellow), or other fluids of specific colors.

In some embodiments, a light-based sensor may include a combination of light transmitters and detectors in the ultraviolet to infrared spectrum to measure the scattering of light by the fluid to measure its turbidity. Serous fluids are typically clear in appearance and low in turbidity. An increase in turbidity, for example, as measured as an increase in the light measured by a photodetector at right angle, may be indicative of white blood cells and microorganisms within the fluid, which may be due to infection.

A light-based sensor may include multiple light sources and receivers. For instance, a single broadband light source may be used in combination with multiple band-specific photodiodes (e.g. red, green and blue). In this way, the absorption/transmission characteristics of the fluid can be measured across as many bands as there are photodetectors present. Similarly, multiple light sources may be utilized in combination with a single broadband photodetector, whereby each light source is turned on successively and the transmitted light measured accordingly by the photodetector. Lastly, light sources and photodetectors may also utilize dynamic filters to allow the emission or detection of specific bands of light in lieu of multiple sources or photodetectors.

In some embodiments, sensors include an impedance sensor typically operated with an alternating current (AC) excitation which may be used to evaluate the user status. An impedance sensor may include an electrode pair, and include an excitation and readout circuit.

In some embodiments, an impedance sensor may be configured to perform AC excitation within a well-defined and constant fluid geometry (constrained by a channel or housing), allowing a normalized impedance (or specific impedance) and admittance to be determined.

In some embodiments, a fluid's impedance may be measured across a range of frequencies (ranging from Hz to MHz) to separate the contribution of individual electrolytes and infer the ionic composition of the fluid. A user condition may be based at least in part on the fluid's ionic composition.

Measured impedance values may be transformed to determine a conductivity (for example, real element of the impedance) of a fluid. Conductivity may reveal a characteristic of the fluid itself, and hence may directly serve a clinical value. For example, conductivity may indicate an analyte's inherent characteristics and composition.

Impedance may be affected by fluid volume and geometry, and thus measured impedance may be used to localize and track particles and bubbles in a fluid channel.

In some embodiments, an impedance sensor may be used to account for a number of artifacts and error sources in bio-signal measurements.

In some embodiments, an impedance sensor may be used to detect a rapid and drastic increase in impedance beyond the range of bodily fluids which may be indicative of the presence of air bubbles in the channel. Air bubbles are a challenge to catheter based measurements as they cause artifacts with readings.

In some embodiments, an impedance sensor may be used to detect a sudden increase in impedance, which may be indicative of a presence and a quantity of non-homogenous substances and particles (e.g., blood clots, fibrin).

In some embodiments, an impedance sensor may be used to detect blood coagulation (typically characterized by a sudden increase in impedance, followed by a slower but sustained increase in impedance), and hence, the presence of blood and risk of channel blockage.

In some embodiments, an array of impedance sensors placed along the channel may be used to detect and track air bubbles, non-homogenous substances, and/or particles as they travel through the channel, using techniques described herein.

In some embodiments, sensors may include amylase sensors.

In use, systems, methods and devices may monitor for trends and changes in physical and chemical biomarkers that may include but are not limited to pH, temperature, fluid flow, pressure, lactate, lactic acid, nitrates, glucose, alkali ions, oxygen, bicarbonate, inflammatory proteins, bacterial proteins and other biomarkers, for example, that are associated or correlated with leakage.

Single sensors or sensor arrays can be placed along the wall of a catheter, inside dedicated lumens, or in an inline device, that enable the device to detect and monitor if a leak is developing.

In some embodiments, a catheter may be used as a carrier for sensors to monitor the internal compartments of the body such as the peritoneal or pleural cavity, without applying any negative pressure. Catheter may be connected to a balloon or a mechanical pump to apply negative pressure to facilitate the drainage of fluid. A catheter may also be connected to a fluid supply such as saline solution to perform therapeutic and diagnostic functions such as dialysis or irrigation.

In some embodiments, multiple sensors may be spaced apart along a length of a catheter. Multiple sensors placed along the catheter, may allow for multiple regions to be sensed and spatial progression of a leak to be tracked.

A catheter may be formed of a tube having a hollow or solid body and made of medical grade materials, such as a suitable polymer. In some embodiments, the catheter may be a flexible substrate.

In some embodiments, a catheter may be formed of a material with low friction.

A catheter may have different designs where the catheter may be cylindrical, rectangular, flat, or T-shaped in cross-section and the catheter may have a single lumen or multiple lumens.

In some embodiments, sensors may be disposed inside reservoirs where fluids may be collected from a user's body. Reservoirs can include elements such as balloons, pumps or other containers that may hold biological fluids. Sensors disposed within a reservoir can be used simultaneously with sensors placed in catheters. This may allow for more sensors to be utilized to determine a variety of different conditions or post-operative complications such as fluid leakage, infection, inflammation or other dangerous complications.

Sensors such as biosensors may be connected to a monitor such as an electronic data acquisition system (DAQ) that may be situated inside or outside a user's body, which may continuously process data obtained from the sensors. The connection can be established via different methods including but not limited to, wires and connectors that may be embedded within the catheter's body or within at least one lumen designed to allow wires and connectors run through them. The connection may also be established wirelessly by transmitting the data obtained in-vivo from biosensors via a transmitting system to a receiver placed outside the body.

In some embodiments, each of multiple sensors are independently in communication with a monitor.

A monitor may have a screen allowing readouts to be directly observed on the device. A monitor may also use various visual or audio queues such as small LEDs or alarm sounds to signal various events.

Data acquired by a monitor can also be communicated to a computer system via wired or wireless media to allow further analysis and visualization. The data communicated may be processed, raw, or summarized.

In some embodiments, data collected by a monitor can be analyzed to identify trends associated with the development of different complications. This may be performed by evaluating single or multiple data sets acquired from one or more sensors over time to diagnose and determine the stage of development of the complications.

Should one or more of the sensors demonstrate biological trends that are associated with surgical leakage, an alarm signal may be sent from the monitor to a computer-based system allowing users to determine the appropriate medical action.

In an example, a slow decrease in local pH could indicate either a small leak or poor blood supply to the wound site. If a simultaneous slow increase in lactate concentration is observed, it may indicate a lack of blood supply (i.e., ischemia). If lactate concentration is steady, it may indicate a slow leak.

In another example, a sharp decrease in pH may indicate a large leak. If the pH returns to its baseline, it may suggest that a wound is healing despite the leak. If the pH continues to drop, or remains low, it may indicate a significant leak that the body may have difficulty recovering from.

Systems and methods disclosed herein may perform monitoring, detection and diagnosis, and prediction. For example, monitoring may present data that is sensed by sensors such as biosensors. Detection and diagnosis may, by way of algorithms, detect a condition in a user and/or make a determination of a diagnosis, such as a leak, what kind of leak it is, and where the leak came from, for example, with an associated confidence level. A prediction may use sensory data to examine different trends and process signals to predict a leak that may occur in the future, for example, with an associated confidence level. As such, embodiments of systems and methods disclosed herein may identify physiological differences between a leak occurring and precursors to a leak.

Systems and methods disclosed herein may be used to perform clinical functions. In an example, a catheter system may be connected to mechanical elements that can apply negative pressure allowing fluid to be drained from a user's body in addition to its diagnostic function. Such clinical function can be both performed at locations in a user body such as inside a GI tract or in a peritoneal cavity.

Techniques for applying negative pressure may include but are not limited to balloons, mechanical pumps, vacuum systems or other devices that can suck fluid, for example, from the body to the outside. In some embodiments, fluid that is being drained may assist in diagnostic application by causing constant fluid flow across sensors. In some embodiments, a clinical function may be performed by pumping fluid into a user's body.

The term “bodily fluid(s)” as used herein may refer to fluids originating from inside the human body, fluids that are excreted or secreted by a body (e.g., blood, gastric juice, and peritoneal fluid), and similar fluids. In extension, the term “luminal fluid” refers to a subset of bodily fluids that exist within inner cavities, intestines, vessels, tubular organs and many other membrane-bound organs such as gastric juices, intestinal fluids, fecal matter, urine, bile fluid, and other similar fluids.

The terms “biomarker(s)” and “aptamer(s)” as used herein may refer to molecules, substances, and chemical or physical properties that can be measured or detected as bio-signals in bodily fluids. They include, but are not limited to, pH, temperature, electrolyte concentration, fluid flow rate, pressure, lactate, lactic acid, nitrates, alkali ions, inflammatory proteins, bacterial proteins, specific cells, molecules, genes, gene products, enzymes, hormones, inflammatory proteins, and glucose.

The terms “biosensor(s)” and “sensor(s)” as used herein may refer to a device or system that detect or react to biomarkers or bio-signals, transducing these signals into measurable electrical signals. Biosensors and sensors utilized herein may include but are not limited to pH sensors, lactate sensors, amylase sensors, lactic acid sensors, glucose sensors, temperature sensors, pressure sensors, enzymatic sensors, protein sensors, biological sensors, ion sensors, electrolyte sensors, impedance sensors, conductivity sensors, flow sensors and other forms of electrochemical and solid-state sensors.

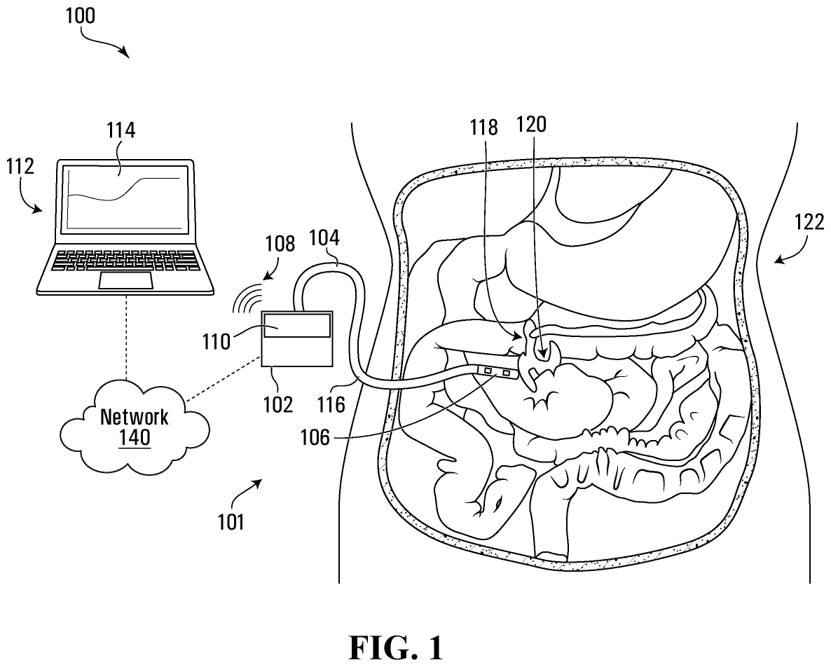

FIG. 1 is a schematic diagram of a system 100 to predict or detect a postoperative complication such as an anastomotic leak in a user, according to an embodiment. System 100 includes a sensor device 101 to sense fluids, having a catheter 104 with sensors 106 attached on it. System 100 also includes a monitor, such as a data acquisition (DAQ) system 102, and an external computing device 112, which may be connected to DAQ 102 by way of a network 140.

System 100 may allow for a user or a patient to be monitored for signs of post-operative leakage, for example, at a medical facility, by having an external monitor 102 placed inside the facility. Further to this embodiment, system 100 may allow a user to leave the facility with a mobile monitor 102 by attaching the monitor to a user's body. System 100 may use visual and audio signals to alarm the user or other individual if a postoperative or surgical complication is detected.

System 100 may include sensor device 101 having catheter 104 and sensors 106 disposed in a user's body, with all other components of system 100 external to the user, locally or at a remote location. Thus, less size, power and functionality may be present at the sensing end of sensor device 101, which may reduce the impact of foreign intrusion on a user's body and also may reduce mechanical stresses on sensor device 101 by virtue of less weight.

In some embodiments, sensor device 101 can enter the body through an incision 116 and can be placed inside abdominal cavity 122 of a user.

Furthermore, sensor device 101 may be designed to be disposable, and cheaper components may be used.

Catheter 104 may be an embodiment of a catheter as described herein.

Sensors 106 may include sensors and biosensors used for detection, for example, of a chemical substance in or from a user's body, and as described herein. As such, sensors 106 may be sized in a suitably small configuration.

In some embodiments, sensors 106 may include flowmeters, as described herein, to measure the volumetric or mass flow rate of liquid or a gas, for example, in or from a user's body.

In some embodiments, sensors 106 may include pH sensors, as described herein, to measure pH of a fluid in or from a user's body.

In some embodiments, sensors 106 may be disposed on catheter 104. In some embodiments, sensors 106 may be disposed on a module that is attached to the end of a catheter 104.

Sensors 106 may monitor the biological fluid surrounding a staple line such as the peritoneal fluid naturally existing in the region. If a failure develops along the staple line 118, sensors 106 may transduce a signal which may be acquired using data acquisition (DAQ) system 102 placed outside a user's body.

DAQ 102 may read a signal that indicates that a leak has developed and a visual 110 and/or audio signal may be relayed.

DAQ 102 may transmit a signal 108 to an external computing device 112, for example, as a wireless signal or over a network 140, and biosignals may be further processed at external computing device 112 and displayed to a user 114. Network 140 may, for example, be a packet-switched network, in the form of a LAN, a WAN, the public Internet, a Virtual Private Network (VPN) or the like.

As illustrated in FIG. 1, system 100 may also include external computing device 112. In some embodiments, external computing device 112 may be located outside of a healthcare institution, and may allow for tele-monitoring of sensor device 101.

In some embodiments, external computing device 112 may be associated with a remote healthcare or medical professional such as a nurse, who may be performing outpatient site visits to a user. Based on the data in signal 108, received, for example from DAQ 102 and associated with one or more users, each having a sensor device 101, a remote healthcare professional may be alerted with a triage for which user to visit first, based on order of urgency of data regarding the monitored status of the user.

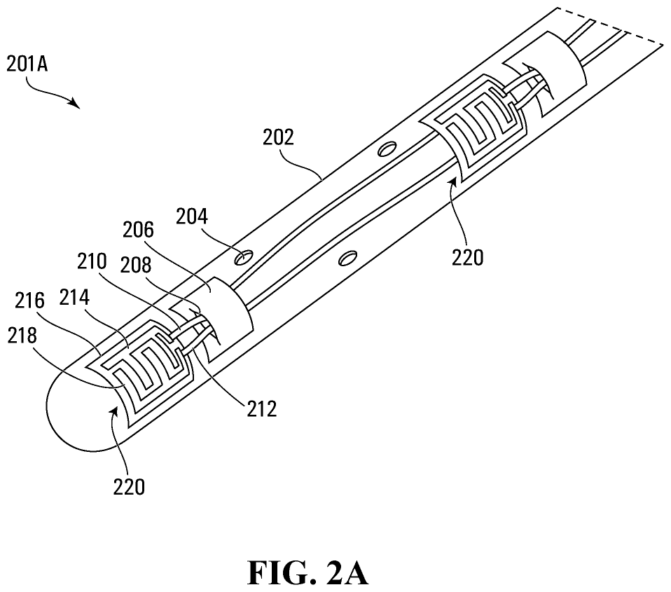

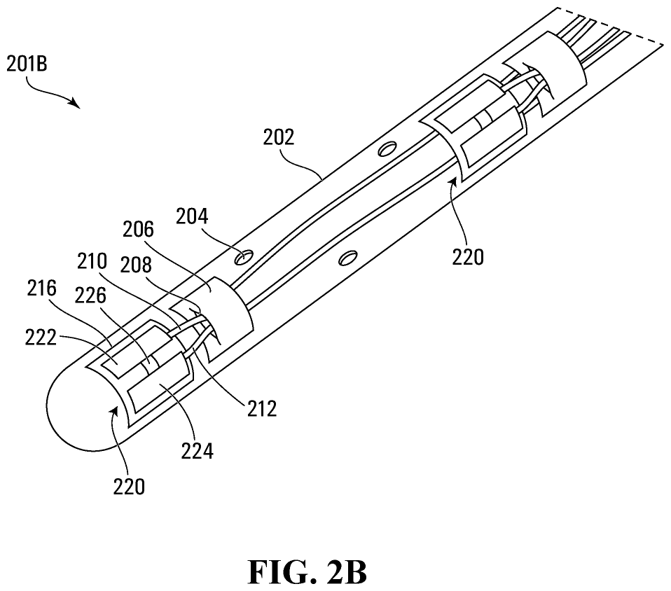

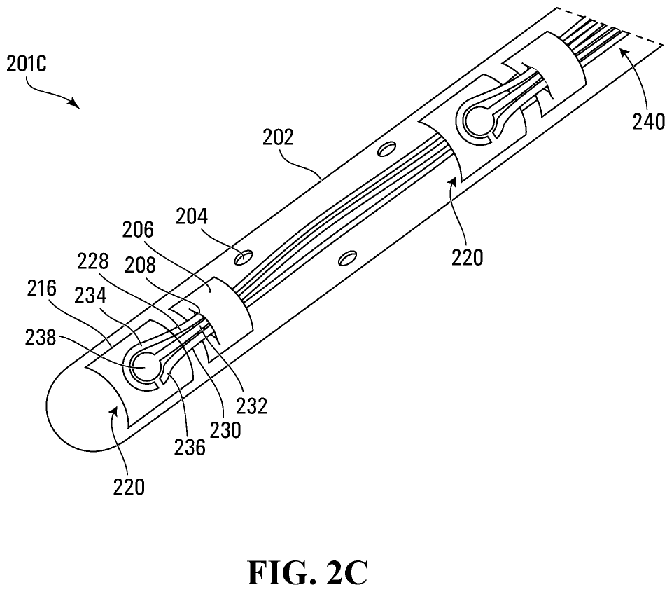

FIGS. 2A-2C illustrate different shapes and forms of sensors 220 that may be utilized across the surface of a catheter 202 of sensor devices 201A, 201B, 201C, respectively. Sensors 220 may include sensors and biosensors as described herein. Sensors 220 may take different forms and perform different functions than those shown here. In some embodiments, sensors 220 can be electrochemical, electromechanical or solid-state in nature.

FIGS. 2A-2C illustrate that sensors 220 may be embedded in the external wall of catheter 202. Sensors 220 may be placed on a flexible substrate 216 or embedded onto catheter 202 body. Further to this embodiment, the system is shown to allow an array of sensors 220 to be utilized.

Sensors 220 are shown connected via two leads 210, 212 for sensors 220 with two terminals or three leads 228, 230, 232 for sensors 220 with three terminals.

Sensors 220 may be electrochemical-based sensors, and have terminals that connect to active, counter, reference or pseudo-reference electrodes depending on the type of sensor being utilized. Sensors 220 may be potentiometric, voltammetric, conductometric, capacitive or amperometric. Sensors 220 may also be solid-state sensors such as field effect transistors (FET) or piezoelectric biosensors. Wire leads may be threaded through holes or perforations 206, 208 into dedicated lumens 240 and exposed to sensors on the catheter surface. Catheter 202 may also have holes or perforations 204 that allow fluid flow into catheter 202.

FIG. 2A illustrates a sensor device 201A having sensors 220 including a system of interdigitated electrodes 214, 218 that may be used for electrochemical sensing, in an embodiment.

In another embodiment, FIG. 2B illustrates a sensor device 201B having active sensing components disposed on the surface of a conductor 226, embodied as sensors 220, with a reference 222 and a counter electrode 224, to enable biosensing of chemical and physical components.

In another embodiment, FIG. 2C illustrates another setup of a sensor device 201C including sensors 220 having a three-electrode based system utilized for biosensing of biomarkers, where the electrodes 234, 236, 238 may act as active, counter, reference and pseudo-reference electrodes and are connected via leads 228, 230, 238.

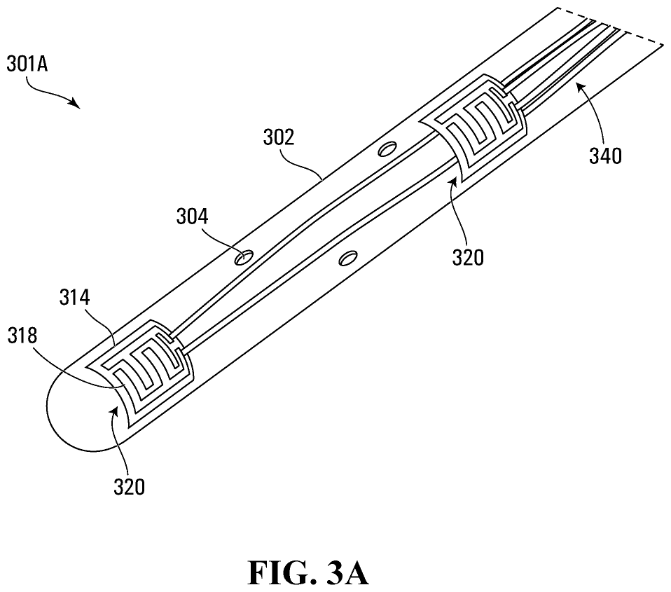

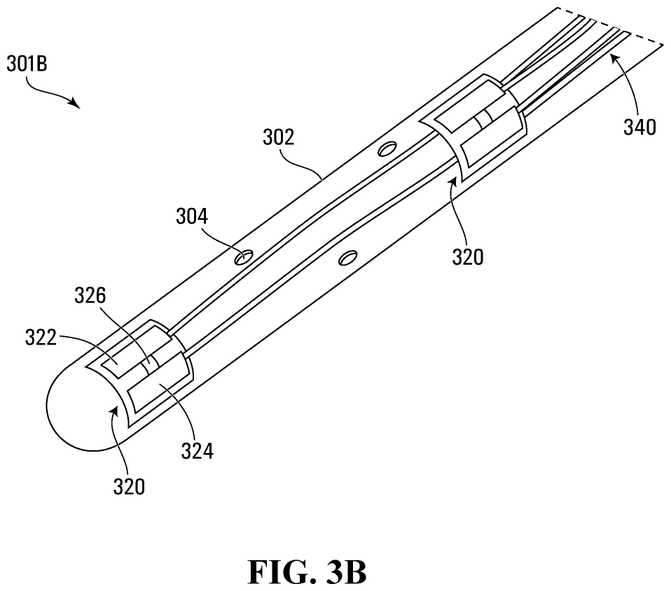

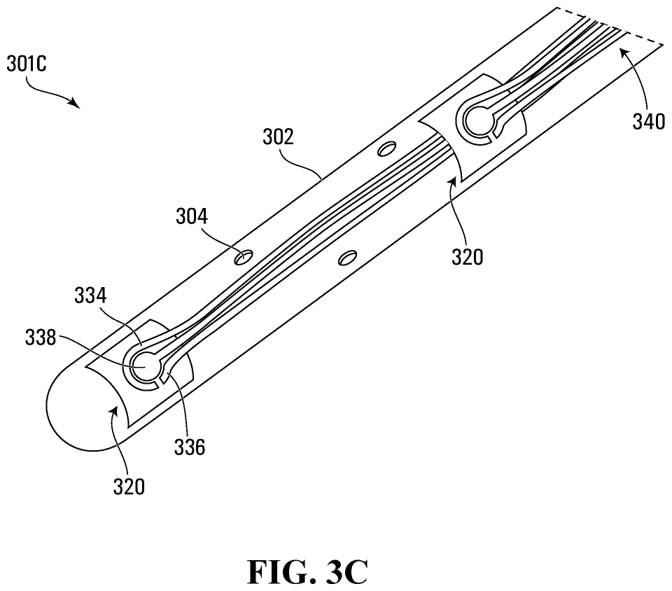

FIGS. 3A-3C illustrate different shapes and forms of arrays of sensors 320 that may be utilized inside dedicated lumens of the catheter 302 of sensor devices 301A, 301B, 301C. Sensors 320 may include sensors and biosensors as described herein.

In some embodiments, sensors 320 are placed within dedicated lumens inside catheter 302. Catheter 302 may have apertures or perforations 304 that allow biological fluid to come in contact with sensors 320 inside of dedicated lumens. Active surfaces of sensors 320 (i.e., where a sensor active component is situated) may be situated to come in contact with fluid that is running through dedicated lumens for other clinical purposes.

Terminals for sensors 320 can connect to active, counter, reference or pseudo-reference electrodes depending on the type of sensor being utilized. For electrochemical-based sensing, sensors 320 may be potentiometric, voltammetric, conductometric, capacitive or amperometric. Sensors 320 may also be solid-state sensors such as Field effect transistor (FET) based biosensors.

Wires 340 may connect to sensors 320 and be disposed inside dedicated lumens, or they may be running inside the same lumens that sensors 320 are active from within.

FIG. 3A illustrates a sensor device 301A, in an embodiment, including sensors 320 having a system of interdigitated electrodes 314, 318 that may be used for electrochemical sensing.

In another embodiment, FIG. 3B illustrates a sensor device 301B, in an embodiment, having active sensing components such as sensors 320 disposed on the surface of a conductor 326 to enable biosensing of chemical and physical components with a reference 322 and a counter electrode 324.

In another embodiment, FIG. 3C illustrates a sensor device 301C, in an embodiment, including sensors 320 having a three-electrode based system that may also be utilized for biosensing of chemical and physical components. Electrodes 334, 336, 338 may act as electrochemical electrodes (active, counter and reference).

FIGS. 2A-2C and 3A-3C show different forms of sensing setups for a sensor device wherein multiple sensors may be placed across the catheter enabling the catheter to actively sense biological fluid in its milieu. Fluid may be sensed by having sensors embedded onto a catheter surface to allow biological fluid around the catheter to be sensed. Sensing may be independent of any other function that the catheter may be performing such as draining fluid out of the body or pumping it into the body. Biological fluid may also be sensed by allowing fluid to flow into the catheter through the multiple perforations on the catheter, for example, perforations 304 as shown in FIG. 3. Sensors may be placed inside dedicated lumens inside the catheter that only house the sensors and their leads without performing any other clinical functions. The sensors inside those lumens may be placed in contact with biological fluids from around the catheter or from those flowing inside the catheter. The sensor configurations shown in FIGS. 2A-2C and 3A-3C illustrate various examples of sensor configurations. In some embodiments, the sensors may have different configurations involving more or fewer leads, different setups, and different form factors.

In the configurations shown in FIGS. 2A-2C and 3A-3C the sensors have been connected in parallel, where every terminal has a dedicated wire. As such, each of the multiple sensors may be independently in communication with a processor, such as a monitor or a computer system as described herein. The sensor array may also allow mapping of the different analytes across catheter 202, 302.

In some embodiments, a sensor device, such as sensor device 201A, 201B, 201C, 301A, 301B, 301C, may be set up such that multiple terminals performing the same function may be connected in parallel where they share the same wire inside the lumen. As an example, all active electrodes may be connected across the body of a catheter such as catheter 202, 302. This catheter design may also allow the user to cut catheter 202, 302 to shorten its length without hindering the functionality (clinical or diagnostic) of the sensor device.

As will be appreciated, utilizing sensors on a catheter as described herein may allow for multiple sensors to be disposed along a body of a catheter. As such, different sensors may be used on a single sensor device, allowing for the analysis of one or more analytes or elements.

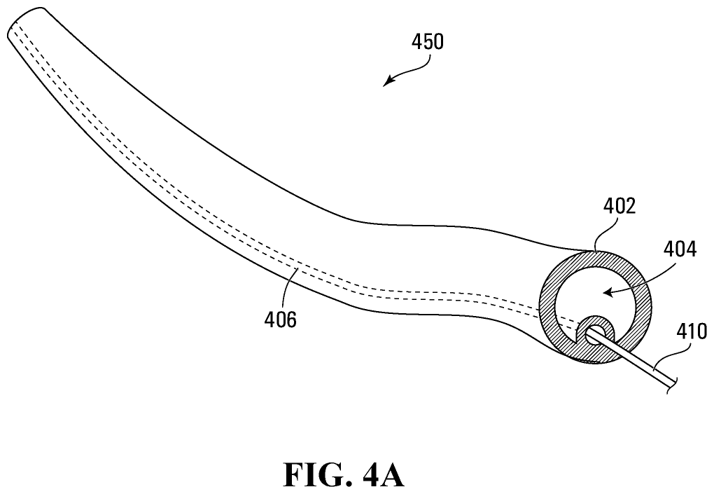

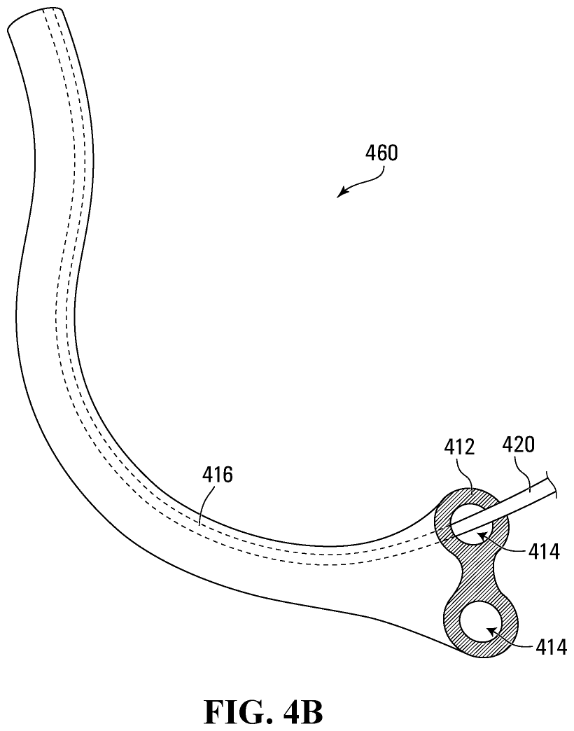

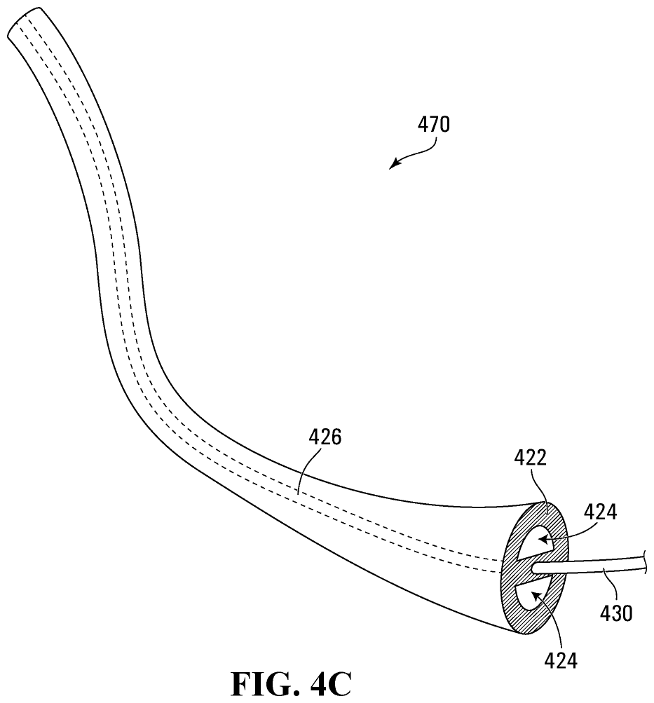

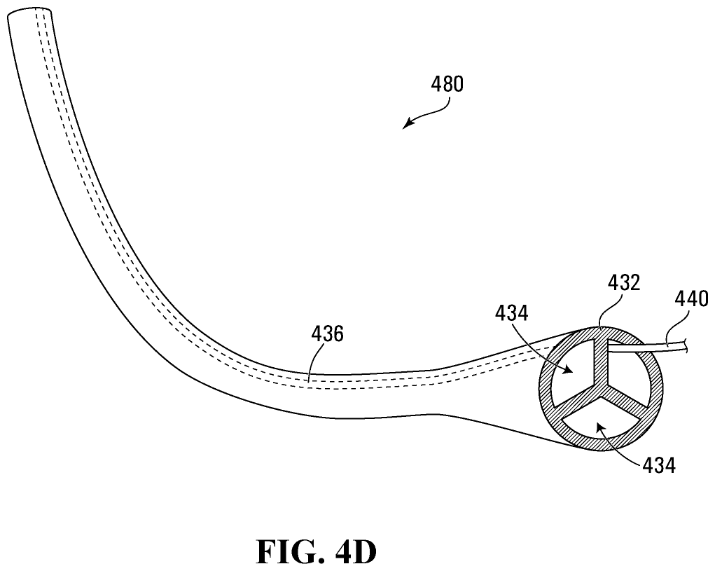

FIGS. 4A-4D illustrate examples of different catheter configurations that may be utilized to enable a clinical and diagnostic application for a sensor device of systems 450, 460, 470 and 480, respectively. Systems 450, 460, 470 and 480 may include sensors and biosensors as described herein.

FIG. 4A illustrates a catheter 402 with a two-lumen system 450, in an embodiment, which would allow one of lumens 406 to be dedicated to wires 410 and their connectors and second lumen 404 may then be utilized to allow fluid to be drained or pumped, for example, to perform a clinical function.

FIG. 4B illustrates a two-lumen system 460 similar to FIG. 4A, in an embodiment. However, in this case the lumen utilized for wires 416 may also be used to allow fluid flow 414 across the catheter 412 and hence is not dedicated for electrical connections 420.

FIG. 4C illustrates a two-lumen system 470 having conductors 430 embedded inside the wall of catheter 422, allowing the two lumens 424 to be used for fluid flow, in an embodiment. Conductors 430 may be wires directly embedded inside catheter wall 426, or the wall of the catheter itself may be manufactured from materials that are conductive.

FIG. 4D illustrates a similar system to FIG. 4C, composed of three or more lumens 436, in an embodiment. Specifically, a three-lumen catheter system is used 480, where one of the lumens 436 has been utilized to allow the wires 440 to be threaded through it, and the catheter's 432 other two lumens may be utilized for fluid flow 434. In the examples illustrated, catheter 432 has a tubular form factor; however it is understood that the form factor of catheter 432 may also be flat, rectangular, tubular, or any combination of those across the body of catheter 432. For example, catheter 432 may have a tubular design on a proximal end of catheter 432 and a flat design on a distal end of catheter 432.

The examples illustrated in FIGS. 4A-4D showcase specific examples that may be utilized. Catheters may be utilized with a wide number and variety of other lumen configurations. Lumens may be designed to only allow wires and connectors to go through them, or to allow only fluids to drain through them, or to allow both wires and fluid to be present within the same lumen.

In some embodiments, multiple lumens may be utilized to calibrate sensors that are being used across a catheter. Calibration may be performed by pumping different fluids into catheter allowing the sensors to calibrate even with the device inserted. Fluids inserted may be specialized fluids containing controlled and specified amounts of one or more biomarkers to allow the biosensors to calibrate. The fluids may also be pH buffers or standard medical solutions that are typically utilized in the clinical environment such as saline solution.

In some embodiments, calibration includes providing a known fluid in contact with sensors to reset baselines for its output. Housing sensors on a catheter such as catheter may allow for the injection of calibration fluids, recording of sensor outputs, and then the fluid being drained back.

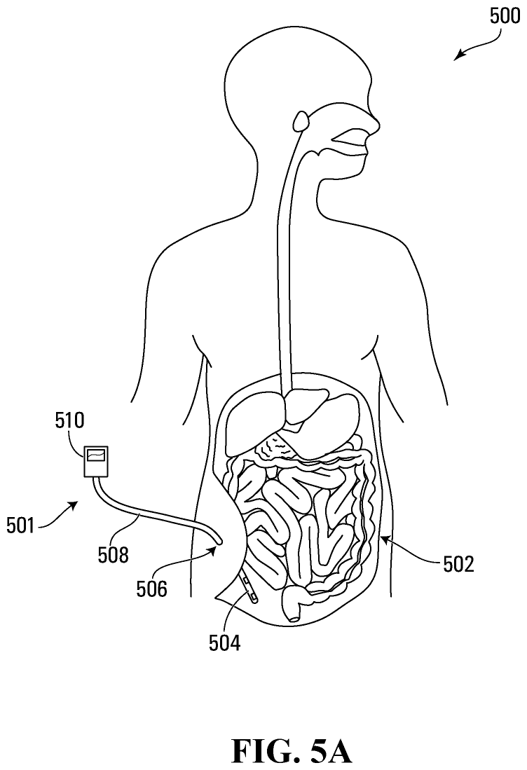

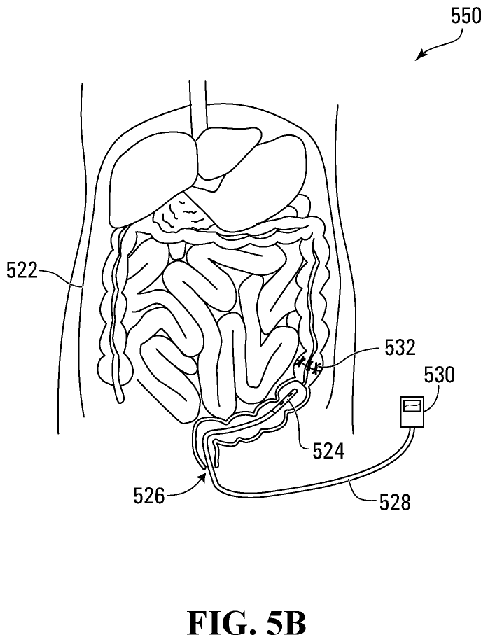

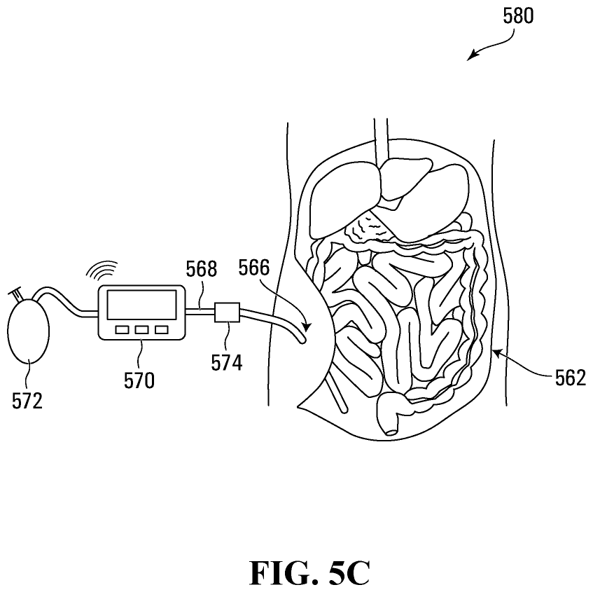

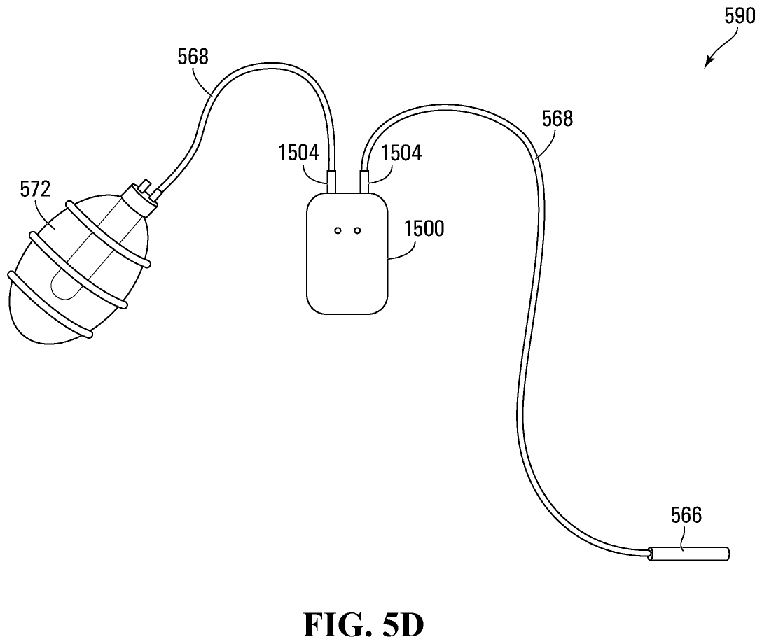

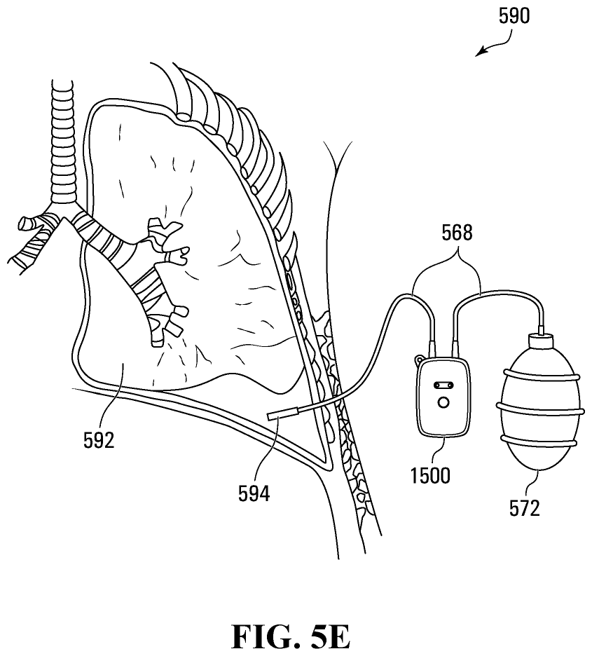

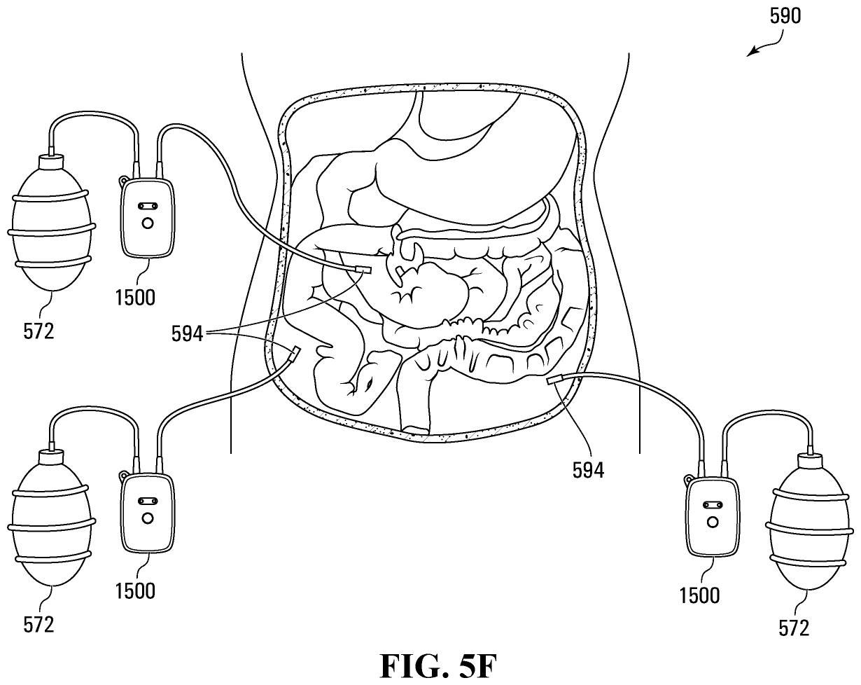

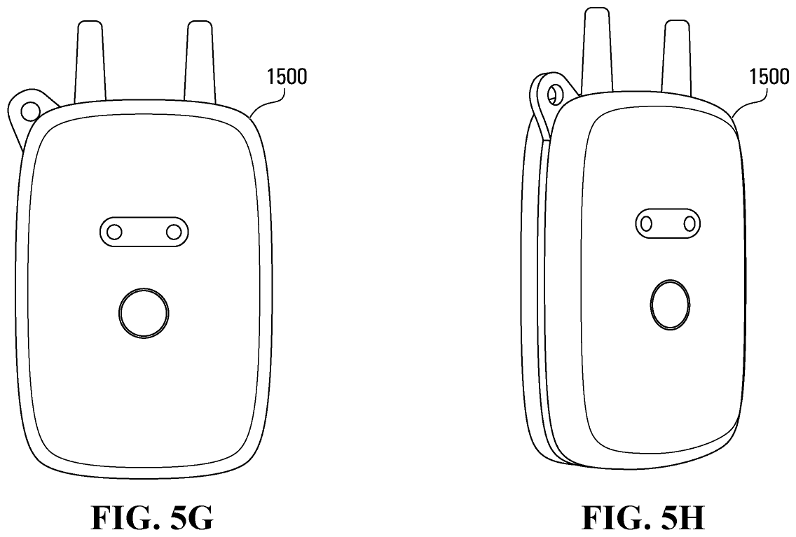

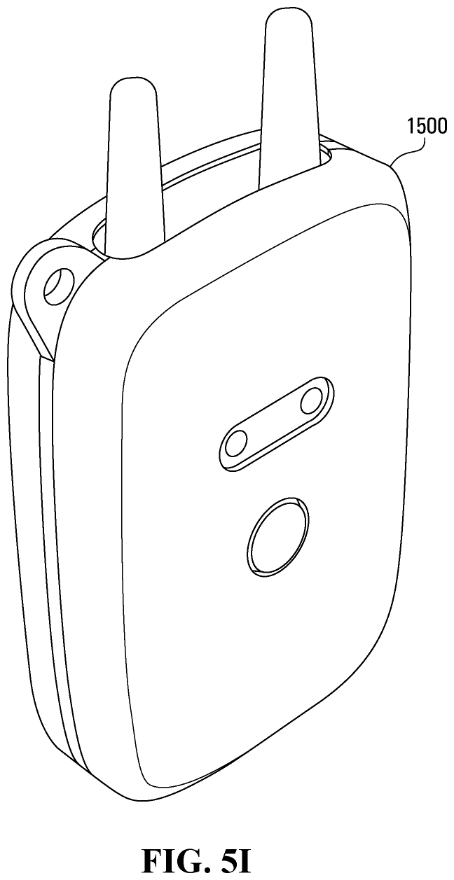

FIGS. 5A-5C illustrate examples of systems 500, 550 and 580, respectively, in abdominal surgeries such as gastrointestinal (GI) surgery. Systems 500, 550 and 580 may include sensors, catheters and monitors, as described herein.

FIG. 5A illustrates a usage of a sensor device 501 whereby catheter 508 may be placed following a laparoscopic surgery through a trocar incision 506, in an embodiment. Sensor device 501 may be placed at different locations in abdominal cavity 502, such as the paracolic gutters, pelvis, proximate to the staple line or somewhere else in the cavity. A diagnostic technique may rely on sensors 504 being in contact with fluid, such as peritoneal fluid which naturally exists in the peritoneal cavity. Due to the biological properties of the fluid, many of the biomarkers may be assessed from the peritoneal fluid and from different locations in the peritoneal cavity. Furthermore, there is constant fluid flow and exchange of biomarkers and substances across the peritoneal cavity.

A system 500 as illustrated by way of example in FIG. 5A may detect different forms of surgical leakages that may arise in abdominal cavity 502. As an example, catheter 508 with sensors 504 may be placed following a laparoscopic abdominal surgery. If a surgical leak develops or shows signs of developing, biomarkers associated with the leak may mix with the peritoneal fluid which then may be detected by the sensor. Further to the example, if a gastric leak (anastomotic leak) is developing due to necrosis along the staple line due to bariatric surgery, the peritoneal fluid can be probed for biomarkers such as lactate, lactic acid, glucose, inflammatory markers, temperature or pH to diagnose the necrosis. When a leak begins, gastric contents may mix with the peritoneal fluid. The peritoneal fluid can then be probed for a multitude of biomarkers and substances such as lactate, lactic acid, glucose, digestive enzymes, food components, inflammatory markers, to determine if a leak is present. If a monitor 510 records signals and trends that are associated with any of those complications an alarm signal can be sent through means such as lights, sounds, cellular messages, Wi-Fi signal, or through other communication channels. An alarm may then be simultaneously communicated to the patient (user), the surgeon, the caregiver or other parties of interest.

A system 550 as illustrated by way of example in FIG. 5B demonstrates the usage of system 550 in gastroenterology applications. As shown in FIG. 5B, catheter 528 may be placed through an orifice 526 to monitor the gastrointestinal tract and detect different forms of surgical leakage or digestive disorders. Hence, system 550 has not been placed inside the peritoneal cavity 522, and thus the fluid that will be analyzed will be different in this application.

Further to this embodiment, FIG. 5B illustrates an example where an anastomosis 532 has been performed, and postoperative monitoring for the patient is performed by sensors 524 placed on catheter 528 in order to determine if a leak develops. Sensors 524 may monitor the intestines for biomarkers and physiological changes such as lactate, lactic acid, inflammatory markers, glucose, digestive enzymes, peristalsis, gas bioproducts, pH, temperature and different biomarkers to identify and determine if a leak is going to develop. If a monitor 530 records signals and trends that are associated with any of these complications, an alarm signal can be sent through means such as lights, sounds, cellular messages, Wi-Fi signal, or through other communication channels. An alarm may then be simultaneously communicated to the patient (user), the surgeon, the caregiver or other interested parties.

A system 580 illustrated in FIG. 5C may detect different forms of complications that may arise in abdominal cavity 562 or other body region of a user, and includes a catheter 568 that penetrates the user's abdomen through wound drain 566 such as a surgical trocar. A reservoir 572 exterior to the user's body collects drainage fluid and may apply negative pressure on catheter 568 to drain fluid from abdominal cavity 562 through perforations in catheter 568, for example, adjacent abdominal cavity 562. Reservoir 572 may be, for example, a bulb, balloon or drainage bag. Fluids can be drained using negative pressure or without any negative pressure being applied to the system.

An inline monitoring device 570 housing sensors, computation and communication modules may monitor fluid as it is drained through catheter 568 to reservoir 572. In some embodiments, inline monitoring device 570 may attach to wound drainage catheters that are typically used to drain fluid for therapeutic (e.g., to relieve pressure) and diagnostic purposes.

As shown in FIG. 5C, in some embodiments, inline monitoring device 570 is exterior to a user's body. Sensors may include, for example, sensors such as sensors 106, sensors 220, sensor arrays 320, sensors 504 or other suitable sensors as described herein, for example, to detect pH, temperature, impedance, conductivity and/or electrolytes in fluid flowing from abdominal cavity 562. Computation and communication modules of inline monitoring device 570 may be embodied as a computing device such as computing device 1000, described in further detail herein.

Inline monitoring device 570 may include one or more inlets and outlets. Catheter 568 is in fluid communication with the inlet of monitoring device 570, and reservoir 572 is in fluid communication with the outlet of inline monitoring device 570. The inlet and/or outlet of inline monitoring device 570 may comply with standard catheter sizes in order to be compatible with existing peritoneal drains. For example, catheter sizes may typically range from 3 mm to 10 mm in diameter (9 FR to 30 FR).

A flowmeter 574 may be installed on a fluid path between abdominal cavity 562 and reservoir 572, for example, in-line with catheter 568 and adjacent inline monitoring device 570 and/or reservoir 572, to measure fluid flow rate. Flowmeter 574 may be used, for example, to detect an obstruction in catheter 568 or to determine if reservoir 572 is at fluid capacity. Flowmeter 574 may be mechanical (e.g. turbine-based), solid-state (e.g. MEMS, thermo-transfer), ultrasonic, or other suitable flow detector. Flowmeter 574 may send signals relating to rate of flow data to inline monitoring device 570, for example, to alert the user that there is no flow of fluid.

In some embodiments, multiple flowmeters such as flowmeter 574 may be installed along catheter 568 or fluid path between abdominal cavity 562 and reservoir 572.

In some embodiments, inline monitoring device 570 may include multiple inlets and outlets to provide multiple independent channels in inline monitoring device 570, through which fluid may flow. These multiple channels may be fed from a single split, or multiple, catheters 568 in contact at or adjacent abdominal cavity 562. As such, multiple channels of inline monitoring device 570 may function in parallel, and each may perform the same or different sensing functions.

Inline monitoring device 570 may be installed in a user immediately after surgery or during the post-operative period.

Conveniently, since it is non-invasive, inline monitoring device 570 may be installed in a user at any time as long as a wound drainage catheter was already implanted. This may be advantageous, since the device may be attached proactively after surgery to detect complications early, after a complication is already suspected to diagnose such complication, or after diagnosis to guide intervention by assessing its efficacy and providing timely feedback to the clinical team. For example, if a post-operative leak occurs and endoscopic intervention is done accordingly to seal the leak, device 570 may monitor drainage fluid to assess the efficacy of such intervention and whether the leak was sealed.

Furthermore, owing to the fact that device 570 is non-invasive and monitors exudate fluids in-vitro, it may employ sensors that are not necessarily biocompatible and may not require sterilization.

System 580 may be used to determine a user condition, such as a clinical condition. Such a condition may be an occurrence of a leak and system 580 may predict a future occurrence of a leak using techniques described herein, based at least in part on data from sensors of inline monitoring device 570 and flowmeter 574.

In use, there may be a latency in a response sensed by inline monitoring device 570, due to the distance from abdominal cavity 562. There may also be a mixing of fluids between abdominal cavity 562 and inline monitoring device 570 where sensing takes place. As such, there may be a smaller signal to be detected, and higher sensitivity sensor hardware may be used.

Since inline monitoring device 570 is sensing at a location that is remote from a surgical site, such as abdominal cavity 562, the fluid being sensed may be mixed and/or diluted, and this may be accounted for at inline monitoring device 570 by way of hardware and/or software.

In some embodiments, software of inline monitoring device 570 may estimate an effect of fluid being mixed before or as it reaches inline monitoring device 570. For example, based on readings of flowmeter 574, the volume and rate of fluid being drained from abdominal cavity 562 may be determined. The volume and rate of fluid movement may be used, in conjunction with other techniques described herein, to determine a user condition, occurrence of a leak, or prediction of a future occurrence of a leak in a user. For example, gastric juice has a low pH. If an increase in flow of fluid is sensed along with a drop in pH, even if the pH is detected at a level that is not as low as would be expected by the presence of gastric juice, a detected increase in flow volume may indicate that the gastric juice is diluted or mixed, and as such, a detected pH level may indicate the presence of material (for example, gastric juice that is leaking) that has a lower pH level than what the current pH reading may otherwise indicate.

Inline monitoring device 570 may also take into account different sensed variables and how they interact with each other. Some sensors of inline monitoring device 570 may not affect the fluid sample that is being sensed. Other sensors of inline monitoring device 570 may affect the fluid sample that is being sensed, for example, by breaking down molecules. One example of this is lactate. Lactate may be broken down in order to measure it. Depending on the flow rate of fluid passing through inline monitoring device 570, a fresh sample fluid may not be available, for example, in the case of an obstruction. Since originally-present lactate may be broken down when previously-measured, inline monitoring device 570 may sense less lactate than is actually present from a user's abdominal cavity. As such, the sensed flow of fluid may be used to account for readings of other sensors.

Inline monitoring device 570 may also, while sensing the fluid, take into account that there is no further diffusion into tissue at the sensing location, as the fluid has left the user's body. Hence, analytes are not replenished and may diminish in concentration due to sensory interaction (for example, molecular breakdown due to a redox reaction).

In some embodiments, monitoring device 570 may also be equipped with visual and audio devices that may signal to a healthcare provider, for example, by way of an audio or visual alarm. In some embodiments, monitoring device 570 may be equipped with wireless transmission systems that may communicate with an external computing device.

In some embodiments, sensors such as sensors 106, sensors 220, sensor arrays 320 or other sensors as described herein may be provided in system 580 inside a lumen of catheter 568. Catheter 568 may be labelled to identify the positions of sensors, such that in use catheter 568 may be position such that the sensors are outside the user's body. The label may provide a visual indicator of the location of sensors (visible through the body of the catheter) so that a person can visually determine if the sensor is external to the user's body.

In some embodiments, fluid may be drawn from abdominal cavity 562 through catheter 568 and inline monitoring device 570 by way of gravity, for example, to a drainage collection bag (not shown). In some embodiments, fluid may be drawn from abdominal cavity 562 through catheter 568 and inline monitoring device 570 by way of capillary action.

FIG. 5D is a schematic diagram of a system 590 including a sensor device embodied as an inline monitoring device 1500, in accordance with an embodiment.

System 590 may be generally similar in structure and components to system 580, including catheter 568, reservoir 572 and wound drain 566.

FIG. 5E illustrates a configuration of system 590, with inline monitoring device 1500 disposed adjacent a thorax, in accordance with an embodiment.

As illustrated in FIG. 5E, in use, system 590 may include a drainage catheter 594 implanted in a pleural cavity 592 for drainage from pleural cavity 592 of a user, for example, following thoracic surgery.

FIG. 5F illustrates an embodiment of system 590 including inline monitoring device 1500, implanted in an abdomen.

FIG. 5H is a front view of inline monitoring device 1500. FIG. 5H is a side perspective view of inline monitoring device 1500. FIG. 5I is a top perspective view of inline monitoring device 1500.

Inline device 1500 may be attached intra-operatively or at any point in time post-operatively, including on patients where drains are implanted post-operatively (e.g. in interventional radiology). Inline device 1500 may be attached preemptively to continuously monitor a patient proactively before a complication is suspected, after a complication is suspected for further monitoring and diagnosis, or during intervention to assess its interventional efficacy and guide further intervention.

In some embodiments, inline device 1500 may attach to a user by means of a hook, loop, clip, hook and loop fasteners (such as Velcro™), or other method to minimize the risk of detachment (e.g., due to patient movement) and the patient burden of holding a device at all times.

In some embodiments, inline device 1500 may attach to the patient's garment near the wound or reservoir by means of a hook, loop, clip, hook and loop fasteners (such as Velcro™), or other method to minimize the risk of detachment (e.g., due to patient movement) and the user's burden of holding a device at all times.

In some embodiments, sensors may be disposed in a channel of an inline device 1500 external to the user's body, anywhere along the fluid's path from a surgical drain to its reservoir. In some embodiments, multiple sensors may be placed on one substrate placed within one system along the length of catheter 568.

In some embodiments, inline device 1500 includes a sensor assembly 1502 having ports 1504 as an input and output in fluid communication with a fluid channel 1506 in sensor assembly 1502.

Sensor assembly may be in communication with a signal conditioning circuit (not shown). The signal conditioning circuit may be a filtering and buffering circuit and include a suitable microcontroller. The signal conditioning circuit may be configured to provide electrical excitation and sensing, for example, for sensing electrodes, magnify weak signals measured using an analog to digital converter (ADC).

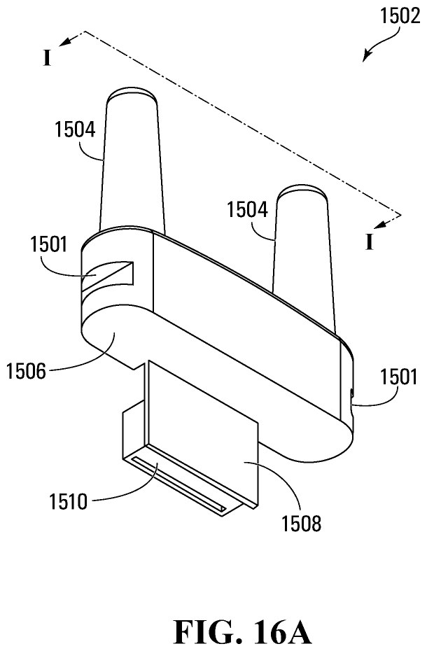

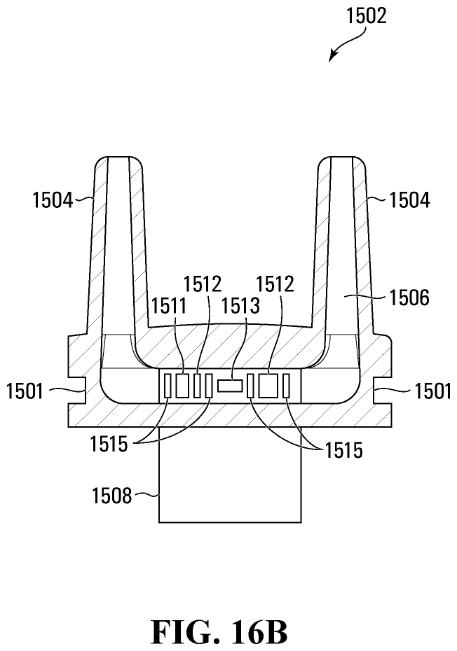

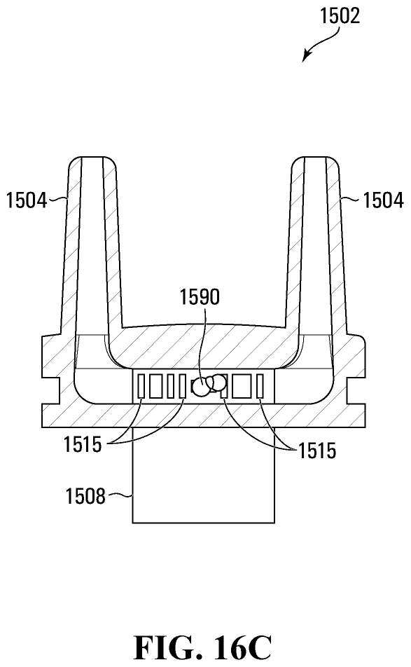

FIG. 16A is a perspective view of sensor assembly 1502 of inline monitoring device 1500, according to an embodiment. Sensor assembly 1502 includes sensors disposed on a substrate 1508, embedded within the fluid channel 1506, and in contact with fluids, as well as air bubbles and particles in fluid channel 1506, in accordance with an embodiment. Sensors of sensor assembly 1502 may include sensors and biosensors as described herein. Sensors may be connectable with other components of inline monitoring device 1500, for example, electronically, by way of an interface providing for a wire connector 1510.

Sensor assembly 1502 may include notches 1501 which can engage, such as by way of a snap fit, with components of inline monitoring device 1500 for attachment to inline monitoring device 1500.

In some embodiments, sensor assembly 1502 may include one or more ports 1504 that interface with a wound or body part of a user and reservoir 572 by way of catheters 568 (inlet and outlet, respectively) and in fluid communication with fluid channel 1508 in sensor assembly 1502 of inline monitoring device 1500, which is embedded with sensors and through which fluid flows.

Ports 1504 may be located in parallel or generally parallel, as illustrated for example in FIG. 5D, to allow parallel or generally parallel attachment of catheters 568, which may minimize the risk of kinks, blockages, and overall device footprint, and may facilitate the attachment of device 1500 to reservoir 572, a patient garment, or an independent hanging structure.

In some embodiments, sensor assembly 1502 may include two ports 1504, or three or more ports 1504, for example, if a user has one or more catheters attached.

FIG. 16B is a cross-section view of sensor assembly 1502 along lines I-I. FIG. 16C is a cross-section view of the sensor assembly of FIG. 16A along lines I-I with a suspended particle 1590 in fluid channel 1506.

Sensor assembly 1502 may include sensors as described herein. The sensors may be similar to sensors 106, 220, 320 and 504, described herein, to measure any of the biomarkers described above such pH, lactate, temperature, amylase, impedance or flow rate.

As illustrated in FIGS. 16B and 16C, sensors in an embodiment of sensor assembly 1502 may include a reference electrode 1511, a pH electrode 1512, a thermistor 1513, a flow sensor 1514 and impedance electrodes 1515.

In some embodiments, sensor assembly 1502 includes one or more light-based sensors as described herein.

Impedance electrodes 1515 may be used to measure the impedance of the fluid by exciting an AC current (typically 1 to 64 kHz) through electrodes and measuring the voltage developed across the fluid, or vice versa. In some configurations, separate electrodes are used for current excitation and voltage measurement in order to isolate the voltage across the fluid only, and exclude the voltage drop across the electrode-fluid interface from the measurement. When impedance is normalized by the channel's geometry, it may be used to calculate a fluid's conductivity. The geometry factor may be calculated empirically using suitable calibration fluids.

A fluid's electrical conductivity can be modeled as the sum of the conductivities of the individual charge carriers (e.g., ions) in solution, whereby an individual charge carrier's conductivity is a product of its molar concentration and molar conductivity. The ionic composition of serous fluids is generally well-controlled to maintain isotonicity, and hence exudate fluid would have a narrow range of conductivity for healthy patients (approximately 9 to 12 mS/cm). However, an exudate fluid's ionic composition's may change (e.g. increase or decrease) due to mixing with luminal fluids (e.g. gastric fluid, fecal matter) that are inherently different (e.g. lower sodium in gastric juice), less controlled and may be affected by a user's diet.

Furthermore, a fluid's conductivity can be affected by its temperature, viscosity and the presence of impurities. Higher viscosity and the presence of impurities typically reduce conductivity due to lower charge mobility, while higher electrolyte concentrations increase conductivity due to the greater number of charge carriers (in this case, ions). Therefore, a decrease in conductivity may be indicative of a luminal leak, particularly if leakage fluid is more viscous or contains solid impurities (e.g., duodenal fluid).

In addition to the natural change in conductivity due to physiological changes in serous fluids or the mixture of luminal fluids into the exudate when complications occur, sensing conductivity may also be utilized as part of a clinical workflow in order to confirm the presence of a leak. This is done by administering a fluid of known conductivity into the organ of concern and monitoring the exudate's conductivity response. For example, if a gastric leak is suspected, a patient may orally administer pickle juice, which has a conductivity of approximately 30 mS/cm. If the conductivity of the exudate's fluid increases accordingly (even if not as high as pickle juice itself), it is likely due to the presence of a gastric leak. Similarly, if a rectal leak is suspected, high-conductivity fluid may be administered anally by means of an enema, and the exudate fluid's conductivity monitored accordingly.

Increases in temperature, on the other hand, increase charge mobility and therefore increase conductivity. This is typically reported as a percentage change in conductivity per degree Celsius. Therefore, a temperature sensor such as thermistor 1513 may be included in conjunction with a conductivity sensor in order to measure the fluid's temperature and report a temperature-corrected value.

Furthermore, by measuring the fluid's conductivity in segments along the channel, a spatial image of the fluid's conductivity to be constructed. The conductivity image of a homogenous fluid would be constant along the channel. On the other hand, if impurities are present (such as, but not exclusively, air bubbles and blood clots), the conductivity image would localize such objects within the channel. This is typically observed as a sharp decrease in conductivity measured between the electrodes that surround an object.

Furthermore, spatial images of the fluid's conductivity may be acquired at a sufficient frequency (depending on the channel size, approximately 1 to 10 Hz) to track the motion of fluid, and impurities in particular, inside the channel. This may be useful to indicate the presence of new fluid in the channel, and hence to enable, increase the accuracy of, or increase the frequency of acquisition of other sensors, amongst other actions. It may also be used to estimate the fluid's velocity, and hence its flow rate. Lastly, the conductivity of the fluid itself, without the effect of impurities, can be measured as the maximum conductivity along any segment of the channel (assuming impurities are not present along the whole channel).

Due to the trauma to organs and tissue that takes place during surgery, drainage fluid in the early periods of recovery is typically contaminated with non-serous impurities such as but not limited to blood, clots, inflammatory markers and proteins, and air bubbles. As the wound heals and the body, initial impurities are drained, and the body reaches homeostasis, the amount of impurities typically decreases such that the exudate appears as a purely serous fluid (typically characterized as pale yellow). The presence of impurities after the initial recovery period (e.g., first day) after surgery may be indicative of a complication. For example, the presence of blood or clots may indicate continuous internal bleeding at the wound site. Similarly, bubbles may be indicative of leakage of gas (e.g., air, methane) from luminal organs. Systems and methods disclosed herein may indicate the presence, size, and quantity of impurities particularly at later stages of recovery.

A flow of fluid through device 1500 may be detected by flowmeter flow sensor 1514. The volume of fluid drained is typically greater in the initial recovery period than it is in later stages (e.g., more than 100 mL/day on average during the first day, less than 100 mL/day on average afterwards). Sustained high volumes of drainage fluid may be indicative of inflammation, infection, or leakages. System 580 may utilize a flow sensor that operates on the principle of mass thermal transfer, on the principle described above, or other types to measure the instantaneous rate of fluid flow. This may be used to calculate the total volume of fluid drained within a period of time and assist in extrapolating the amount of fluid expected in the near future. Systems and methods disclosed herein may alert if the flow rate is higher than expected after the initial recovery period (as determined by either the clinical team or a default value based on research) or if there is a sudden increase in flow rate.

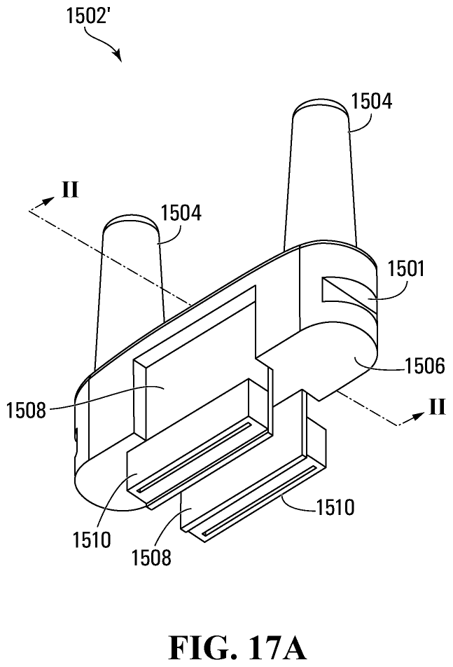

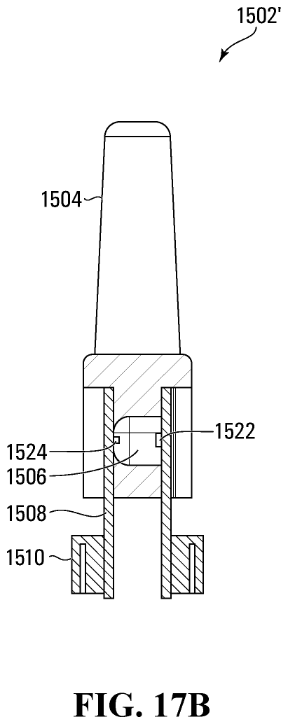

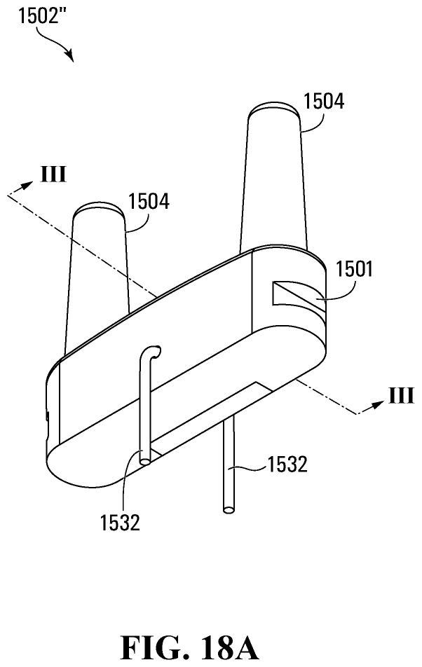

FIG. 17A is a perspective view of a sensor assembly 1502′ having two substrates 1508 including light-based sensors, in accordance with an embodiment. FIG. 17B is a cross-section view of sensor assembly 1502′ along lines II-II.

Sensor assembly 1502′ may be generally similar in structure and components to sensor assembly 1502, including notches 1501, ports 1504, fluid channel 1506, two of substrates 1508 and two of wire connectors 1510.

Sensor assembly 1502′ may include a light-based sensor, as described herein.

In some embodiments, as illustrated in FIG. 17B, a light-based sensor of sensor assembly 1502′ includes light sources 1522 and photodetectors 1524 placed on separate substrates 1508 and embedded within fluid channel 1506 across from each other to measure light transmittance through the fluid passing through fluid channel 1506, in accordance with an embodiment.

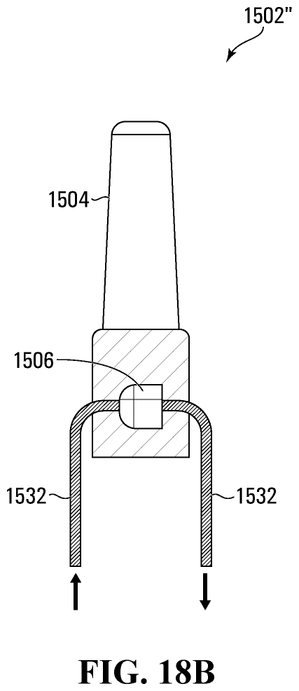

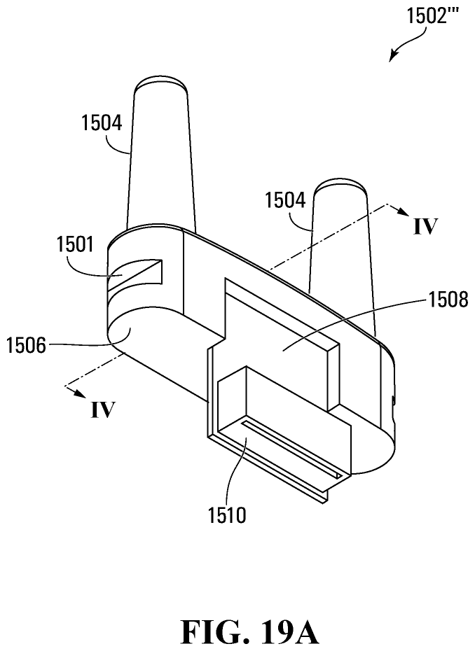

FIG. 18A is a perspective view of a sensor assembly 1502″ having a fibre optic light-based sensor, in accordance with an embodiment. FIG. 18B is a cross-section view of sensor assembly 1502″ along lines III-III.

Sensor assembly 1502″ may be generally similar in structure and components to sensor assembly 1502 and sensor assembly 1502′, including notches 1501, ports 1504 and fluid channel 1506.

Sensor assembly 1502″ may include a light-based sensor, as described herein.

As illustrated in the embodiment of FIG. 18B, a light-based sensor of sensor assembly 1502″ includes fiber optics for light transmission and receipt. In particular, light is guided into the channel through a fiber optic 1532 and received across the channel by another fiber optic 1532 to measure light transmittance through a fluid in fluid channel 1506, in accordance with an embodiment.

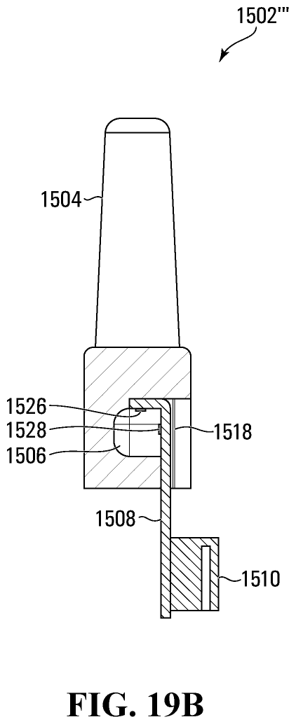

FIG. 19A is a perspective view of a sensor assembly 1502′″ having a light-based sensor, in accordance with an embodiment. FIG. 19B is a cross-section view of sensor assembly 1502′″ along lines IV-IV.

Sensor assembly 1502′″ may be generally similar in structure and components to sensor assembly 1502, sensor assembly 1502′ and sensor assembly 1502″, including notches 1501, ports 1504, fluid channel 1506 and wire connector 1510.

Sensor assembly 1502′″ may include a light-based sensor, as described herein.

In some embodiments, substrate 1508 of sensor assembly 1502′″ includes rigid substrate sections as well as a flexible substrate 1518 at a bending region, as illustrated in FIG. 19B.

As illustrated in FIG. 19B, a light-based sensor of sensor assembly 1502′″ includes a light source 1526 and a detector 1528 disposed on a single rigid-flex substrate 1508 and embedded within fluid channel 1506 at right angle to measure light scatter by fluid in fluid channel 1506, in accordance with an embodiment.

In some embodiments, light source 1526 and photodetector 1528 may be placed at right angles to measure the fluid's turbidity (i.e., the scattering of light by the fluid).

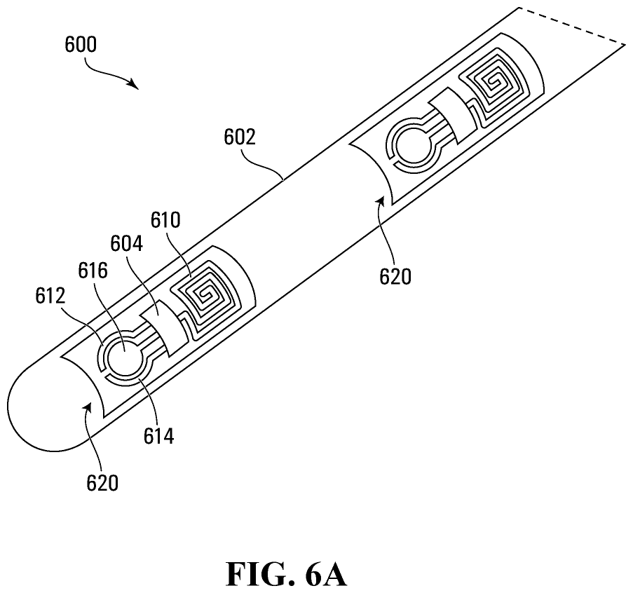

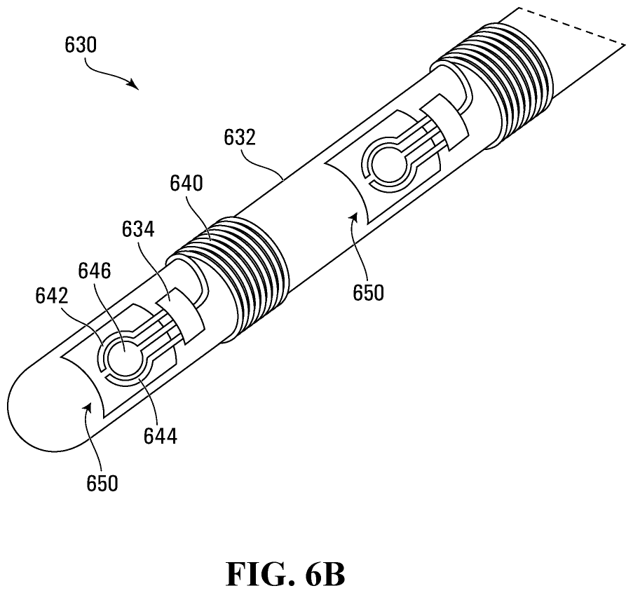

FIGS. 6A-6B illustrate embodiments of a sensor device where the catheter may perform its diagnostic application without the need to have wires connecting the sensor to the monitor and without the need to have dedicated lumens for the wiring, an ay include sensors as described herein. The detection system illustrated may perform the sensing functionality, acquire raw signals and transduce them so that they can be transmitted to an external receiver. The system illustrated may require a power module which may be achieved by having a battery installed on the system, through a power harvesting solution or inductive power transfer. In addition, the sensors may not need the monitor to be placed close to the patient. Furthermore to this embodiment, the system may store the data readings and transmit data only when the monitor is within communication distance, allowing the user (patient) to not carry an additional device. Further to this, the user may not need a monitor, and the healthcare provider may use the monitor to collect the data during hospital visits.

FIG. 6A, illustrates a system 600 where a catheter 602 has an array of sensors 620, an integrated circuit 604 and a trace antenna 610 to transmit the signal. The antenna may alternatively be a conformal antenna. The integrated circuit 604 may include a microcontroller to process the data and connect to a battery. It may also incorporate a transceiver to send data wirelessly through the antenna. Furthermore to this embodiment, a three-electrode system is shown where an active electrode 616, a reference electrode 612 and a counter electrode 614 are shown. The system can be designed such that the sensors can collect the data and the integrated circuit can process the data so that it can be wirelessly transmitted to a receiver external to the body.

FIG. 6B, illustrates a system 630 where a catheter 632 has an array of sensors 650, an integrated circuit 634 and a helical antenna 640 to transmit the signal. The system can be designed such that the helical antenna 640 may be embedded inside the wall of the catheter allowing it to align with the design of the catheter. The integrated circuit 634 may have a microcontroller to process the data and connect to a battery. It may also incorporate a transceiver to send data wirelessly through the antenna. Furthermore to this embodiment, a three-electrode system is shown where an active electrode 646, a reference electrode 642 and a counter electrode 644 are shown. The system can be designed such that the sensors can collect the data and the integrated circuit can process the data so that it can be wirelessly transmitted to a receiver external to the body.

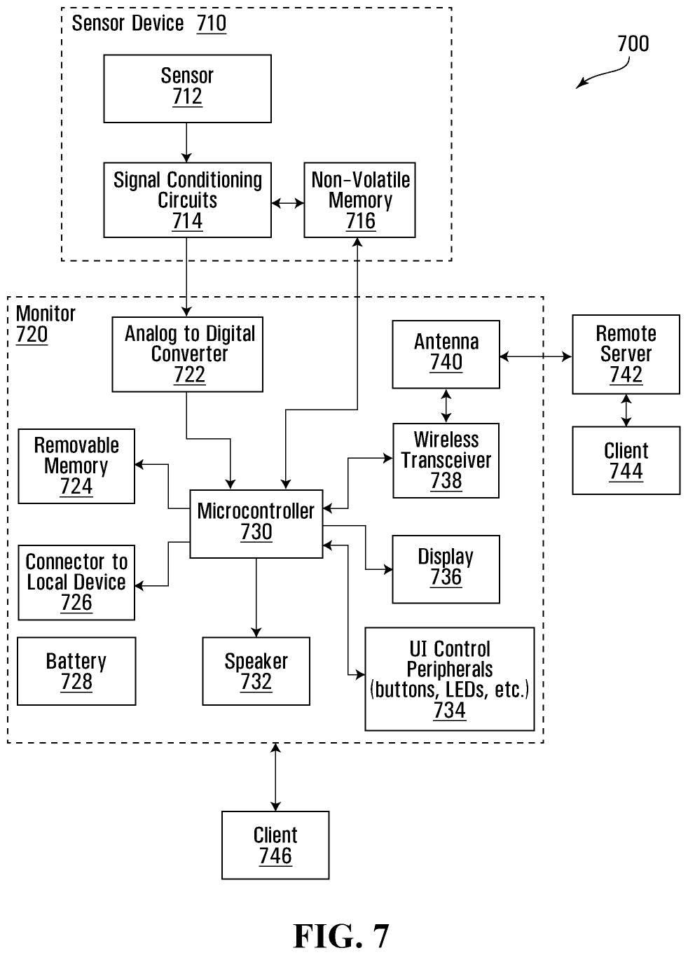

FIG. 7 is a schematic diagram of an embodiment of a system 700 that may be utilized to monitor the status of a sensor device 710 and relay a signal to another device or an end user. In one embodiment of the system, sensor device 710 includes a catheter and adapter having an array of sensors 712 with at least one sensor being used to monitor the status of a patient following a surgery.

Components of systems described herein, such as system 590, may be incorporated into system 700. It will be understood that sensor device 710 may be embodied as any sensor device as described herein, and monitor 720 may be embodied as any monitor or DAQ as described herein. In some embodiments, sensor device 710 and monitor 720 may be embodied as inline monitoring device 1500. Sensors 712 may be any sensors as described herein.

System 700 may utilize a single sensor 712 to perform the monitoring, or it may utilize a system of multiple sensors to monitor the patient. Further to this embodiment, the system may utilize a single type of sensors, or it may use different types of sensors to sense different biomarkers, as described herein.

Sensors 712 may be connected to signal conditioning circuits 714. Circuits 714 may perform different functions such as buffering, filtering, amplifying, encrypting and converting the signal so that it can be read by an analog to digital convertor (ADC) 722.

Signal conditioning circuits 714 may also be connected to a non-volatile memory unit 716 to access calibration data and calibrate sensor measurements.

ADC 722 and signal conditioning circuits 714 may either be placed on the catheter, in the monitor 720, or in an adapter attached to the catheter. In some embodiments, sensor device 710 is connected to monitor 720 by a wired connection.

System 700 may connect to an ADC 722 housed inside the monitor 720, which may then connect to a microcontroller 730. Microcontroller 730 may store the signal on a removable memory 724. Data stored may include raw signals, processed signals, and meta-data about the user (patient) such as their age, gender, medical history, type of operation. The microcontroller may also use this information while monitoring and analyzing signals to determine whether the patient suffered a post-surgical leakage. Removable memory 724 may take different forms, such as secure digital (SD) cards, flash memories, floppy disks, optical disks or other suitable forms of removable memory.

Monitor 720 may also have a connector 726 that may allow for data transfer to or from an external host, charge the system, control an external device, or flash the firmware of the system. Monitor 720 may also contain a power source such as a battery 728 to power the system.

Monitor 720 may also have components that can relay signals directly to the user. Further to this embodiment, the system may utilize components such as speaker 732, a display 736, lights 734 and other elements that may be used to communicate with the user 746, or a computing device associated with user 746. The system can signal an alarm when early signs of a complication arise. Similarly, the system may also signal an alarm if a complication or a leak have been confirmed. Different alarms may also be signaled for different complications. A user 746, such as a healthcare provider, may be provided extra information related to the alarms by looking at the visual feedback from the lights 734 or the display 736. There also may be audio queues that can be signaled via the speaker 732. User 746 may also be able to further access detailed information related to the data captured by the sensors by accessing the removable memory 724 or via the connector 726.

Monitor 720 may be equipped with a wireless transceiver 738 and an antenna 740 enabling wireless monitoring for the patient. The system may allow wireless monitoring over short distances by using systems such as Wi-Fi, Medical Body Area Networks (MBANs), Bluetooth, Zigbee, Near field communication (NFC), Infrared transmissions or other short-range network protocols. The system may also be setup to communicate over long range by utilizing systems such as cellular networks, low-power wide-area network (LPWAN), Lora, or other long-range network protocols. This may thus allow the system to function in the hospital local setting or as a home monitoring device for the users. The data communicated wirelessly may be relayed to a remote server 742 which can be accessed by user 744.

Remote server 742 may include one or more computer servers and may include local, remote, cloud based or software as a service platform (SAAS) servers.

Monitor 720 may be equipped with software that can display the data acquired from the sensors. The monitor software may also display processed data that has been obtained from the sensors. The monitor software may also display a simple message alerting the patient to the status of the complication that may be happening or a complication that may arise.

In some embodiments, system 700 may be designed to be completely wireless without a wired connection between the sensor device 710, and the monitor 720. Where all of the components may be integrated into one device and placed on the catheter as illustrated in FIG. 6A and FIG. 6B. In which case the sensor device 710 may be expanded to included elements such as the antenna 740, wireless transceiver 738, microcontroller 730, power solution and other components to complete the system.

In another embodiment, a computing device may be used to access the data collected and stored inside monitor 720. The software utilized on the computing device may be used to access raw or processed data from the device. The software may further be set up such that the user may collect information regarding the algorithms and the calculations performed in order to determine the clinical status of the patient. The software may also be utilized to determine the clinical status of the patient or to display information obtained from the sensors.

In some embodiments, data may be stored locally or remotely. It may be encrypted to abide by security regulations set forth by government and regulatory boards to protect patient safety. Encryption may be done prior to data processing on the microcontroller 730. Encryption may also be done on data being inputted into the system.

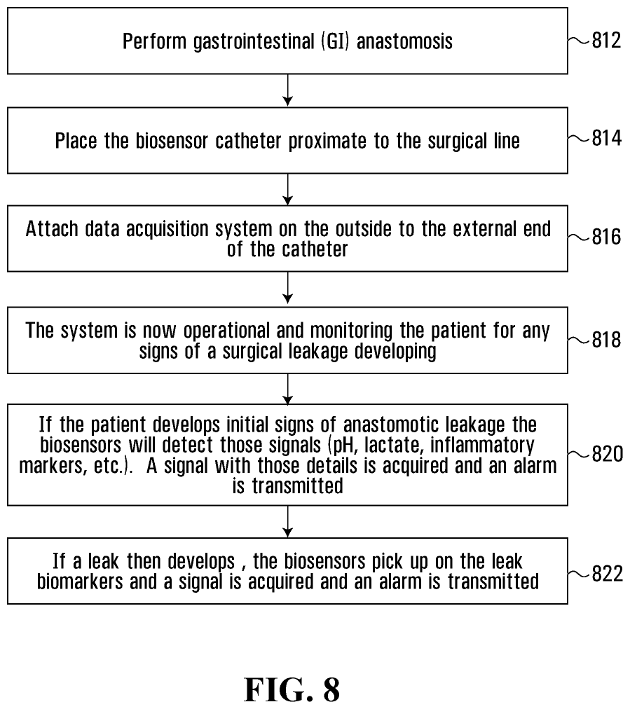

FIG. 8 illustrates a process flow chart for an embodiment addressing a use case for the device. The surgeon would first perform an anastomosis 812 in the gastrointestinal (GI) tract, such as at the stomach, small intestine, large intestine, colon, or rectum of a user. The surgeon may then surgically place a catheter with biosensors 814 at an area proximate to the surgical line. The surgeon may place the catheter in a nearby cavity where fluid may collect, deep in the pelvis, or somewhere else in the peritoneum so that it is in contact with the peritoneal fluid. On the distal end of the catheter (external part), the surgeon may then attach the monitor to establish a connection with the biosensors on catheter 816. The system may then monitor the fluid the peritoneal fluid that exists in the cavity allowing the system to monitor the patient 818.

If the user (patient) develops early signs of leakage 820, the biosensors would be able to monitor the milieu changes in the fluid and identify different biomarkers that may be associated with a developing leak. Depending on the nature of the reading obtained from the biosensors, the monitor may alert the user informing the user to seek immediate medical attention. The monitor may also continue to collect information in order to identify further trends of complications as they develop. If a leak does develop 822 and luminal content does leak into the peritoneum, the monitor would alert the user of the situation and inform them to seek medical attention immediately as well as inform on the nature of the complication.

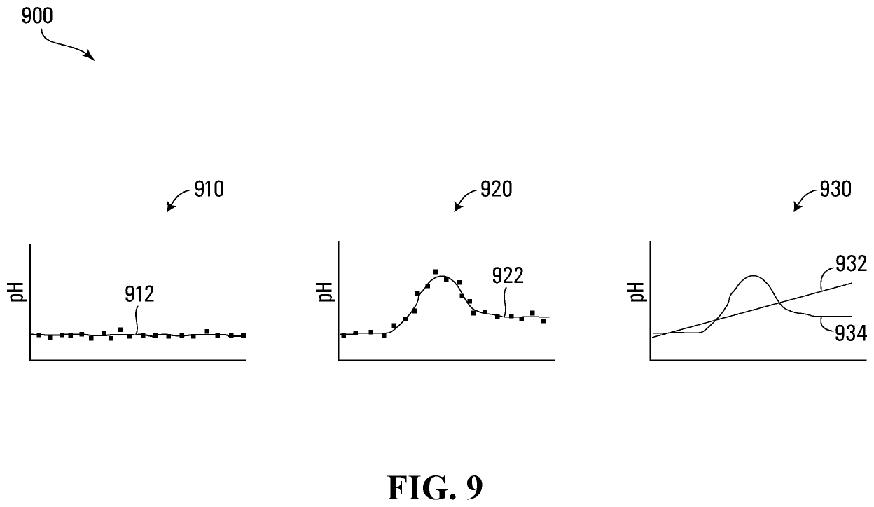

FIG. 9 depicts biosensor plots that have been captured over a period of time, for example, by system 700. Specifically, in this embodiment, three pH curves are shown to be captured over a period of time 900, after a catheter (with biosensors) has been placed inside the peritoneum of a user. Hence the sensors are monitoring peritoneal fluid in the body. In the first curve 910, we see the steady state curve for the biosensors. The blue squares show the independent data points that have been captured, and the solid line 912 shows the data that is being processed by the monitor. In the first curve 910, it may be possible to extrapolate based on the steady state that there is no significant change that could be indicative of a complication. This is typically seen as the region typically has a constant reading, in addition to the fact that the body is very good at regulating pH.

The second curve 920 shows a different trend where the initial readings of the curve 922 show a steady state reading followed by a rise and then another steady state reading. This is abnormal compared to the initial reading shown 910. The monitor may then process the data, to create a new curve 930 with a trend line 932. The data shown in 934 show that the general trend is an increase in pH, which may be associated with different forms of leakage or infection. The first data points the system may analyze are the steady state readings and where they settle to determine the pH of the peritoneum, allowing the determination of a number of clinical factors. Another data point that may be further analyzed to predict the clinical status of the patient is the full width at half maximum (FWHM) to determine if a leak is happening, the type of leak and its development. The system may also further analyze another data point which is the rate of change of pH during the rise and fall peak to determine factors such as the leakage cause, rate of leakage, type of leakage, location of the leak, and when the leak happened.

Further to this embodiment, the data can be accessed by having the data displayed on the monitor directly, or the data may be accessed by using the local connector placed on the monitor 726. The data may also be accessed remotely using the remote server 742, allowing the healthcare provider to monitor the patient without the need for the patient to be available at the medical facility.

In some embodiments, microcontroller 730 of monitor 720 may be embodied as a computing device such as computing device 1000. In some embodiments, monitor 720 may be embodied as a mobile computing device.

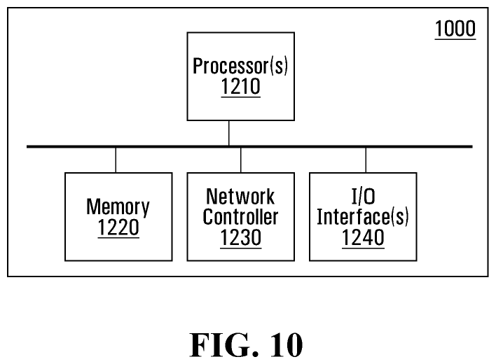

FIG. 10 is a high-level block diagram of a computing device 1000, in an example. As will become apparent, computing device 1000, under software control, may receive data such as biosignal data for processing by one or more processors 1210 to analyze the signal data. Processed signal data may be displayed, for example, on display 736, or communicated over a network to another device such as remote server 742.

As illustrated, computing device 1000 includes one or more processor(s) 1210, memory 1220, a network controller 1230, and one or more I/O interfaces 1240 in communication over bus 1250.

Processor(s) 1210 may be one or more Intel x86, Intel x64, AMD x86-64, PowerPC, ARM processors, TI MSP430 or the like.

Memory 1220 may include random-access memory, read-only memory, or persistent storage such as a hard disk, a solid-state drive or the like. Read-only memory or persistent storage is a computer-readable medium. A computer-readable medium may be organized using a file system, controlled and administered by an operating system governing overall operation of the computing device.

Network controller 1230 serves as a communication device to interconnect the computing device with one or more computer networks such as, for example, a local area network (LAN) or the Internet. Computing device 1000 may communicate with non-volatile memory 716 at sensor device 710, remote server 742, and a computing device associated with user 746, for example, by way of network controller 1230.

One or more I/O interfaces 1240 may serve to interconnect the computing device with peripheral devices, such as for example, keyboards, mice, video displays, and the like. Such peripheral devices may include a display of device 1000. Optionally, network controller 1230 may be accessed via the one or more I/O interfaces.

Software instructions are executed by processor(s) 1210 from a computer-readable medium. For example, software may be loaded into random-access memory from persistent storage of memory 1220 or from one or more devices via I/O interfaces 1240 for execution by one or more processors 1210. As another example, software may be loaded and executed by one or more processors 1210 directly from read-only memory.

In some embodiments, computing device 1000 may be an embedded system or microcontroller, including a processor, memory, and input/output (I/O) peripherals on a single integrated circuit or chip, to perform the processes and store the instructions and data described herein. In an example, computing device 1000 may be a microcontroller such as an Arduino board and associated software system.

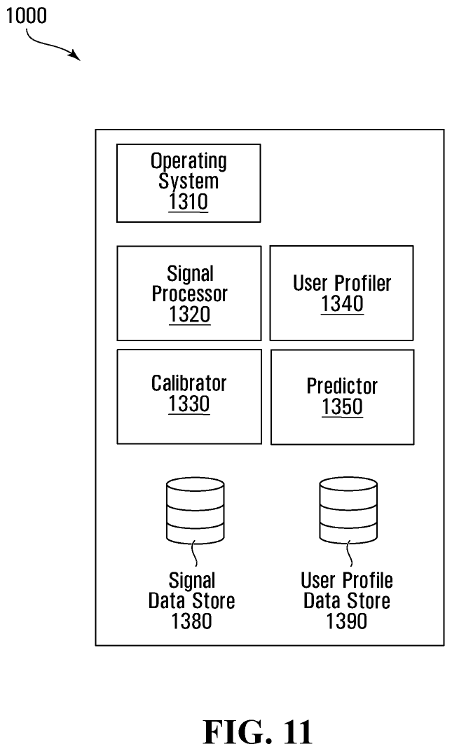

FIG. 11 depicts a simplified organization of example software components and data stored within memory 1220 of computing device 1000. As illustrated, these software components may include operating system (OS) software 1310, signal processor 1320, calibrator 1330, user profiler 1340, predictor 1350, signal data store 1380 and user profile data store 1390.

Operating system 1310 may allow basic communication and application operations related to the mobile device. Generally, operating system 1310 is responsible for determining the functions and features available at device 1000, such as keyboards, touch screen, synchronization with applications, email, text messaging and other communication features as will be envisaged by a person skilled in the art. In an embodiment, operating system 1310 may be Android™ operating system software, Linux operating system software, BSD derivative operating system software, iOS™ operating system software, or any other suitable operating system software. In embodiments in which an Android operating system platform is in use, software components described herein may be implemented using features of a framework API (Application Programming Interface) for the Android platform.

Signal processor 1320 receives signal data, for example, from signal conditioning circuits 714 that have been measured from sensors such as biosensors as described herein.

In some embodiments, signal processor 1320 may process signal data into a format for use by other software. This may include metadata such as calibration coefficients and sampling parameters. Sensory data may be compressed, encrypted, or pre-calibrated, and may be stored in binary, text, or other formats.

In some embodiments, signal processor 1320 may operate to contextualize received signals, for example, pH changes. For instance, sensor outputs may drift slowly over time. Such drift may be corrected in software if sensors are modeled well, or if constraints of a significant change (e.g., amplitude, rate of change, etc.) are known.

Signal data, processed or unprocessed, may be stored at signal data store 1380.

Signal processor 1320 may determine a user condition based at least in part on sensor data detected by signal sensors. In some embodiments, a user condition may be based at least in part on a user profile, discussed in further detail below.

In some embodiments, a user condition may be based at least in part on the relative admittance (amplitude and phase) across a range of frequencies, as previously correlated with user conditions.

In some embodiments, a user condition is based at least in part on a pH level as measured in the user's body or in drainage fluid outside of the patient's body.

In some embodiments, a user condition is based at least in part on the change in pH level detected in the patient's body or in drainage fluid outside of the patient's body.

In some embodiments, a user condition indicates a presence of a fluid in the user's body.

In some embodiments, a user condition is based at least in part on an increase or decrease in the amount of fluid drained from the user's body.

In some embodiments, a user condition is based at least in part on multivariate statistical procedures that transform data from multiple sensors into alternative subspaces that facilitate characterization of fluids and identification of their constituents.

In some embodiments, a user condition is based at least in part on multivariate linear transformations from multiple sensors, such as Principal Component Analysis (PCA), that facilitate the characterization and identification of fluids and their constituents.

In some embodiments, a user condition is based at least in part on the cross-correlation of sensory data with pre-identified characteristic curves for various user conditions.

In some embodiments, a user condition is based at least in part on the absolute output of a biosensor such as pH; impedance; conductivity; lactate concentration; amylase concentration; light absorption or transmission; or other biosensors.

In some embodiments, a user condition is based at least in part on the rate of change measured by a biosensor such as pH; impedance; conductivity; lactate concentration; amylase concentration; light absorption or transmission; or other biosensors.

In some embodiments, a user condition is based at least in part on the cross-correlation of biosensor output with pre-existing trends for user conditions, using biosensors such as pH; impedance; conductivity; lactate concentration; amylase concentration; light absorption or transmission; or other biosensors.

In some embodiments, a user condition is based at least in part on output of all sensors of a sensor device, including but not limited to a sensors as disclosed herein.

In some embodiments, a user condition is based at least in part on a change in sensed values between the multiple sensors.

In some embodiments, signal processor 1320 may monitor a primary condition, such as pH, as well as a secondary conditions, such as detecting temperature for detecting a fever of the user. A secondary condition may be considered in combination with a primary condition, by predictor 1350, described in further detail below. In some embodiments, a primary and secondary condition may be assessed on the basis of time of the conditions, for example, time between a primary condition occurring and a secondary condition occurring.

In some embodiments, signal processor 1320 may evaluate various criteria may be set to prompt alerts, for example, to a caregiver or a healthcare professional. Criteria may be defined by a user.

In some embodiments, a criteria may be defined as detection of an air bubble, for example, by an impedance sensor as described herein. When the user-defined criteria is detected by an appropriate sensor, an alert is sent to a caregiver or healthcare professional. Since bubbles are not typically present after the initial drainage period (approx. 1 day), the presence of bubbles may be indicative of gas leakage through an anastomotic leak.

In some embodiments, a criteria may be defined as a percentage of air bubbles relative to fluid increases above a threshold, for example, as detected by an impedance sensor as described herein. When the criteria is detected by a suitable sensor, an alert is sent to a caregiver or healthcare professional. Since bubbles are not typically present after the initial drainage period (approximately one day), the presence of bubbles may be indicative of gas leakage through an anastomotic leak.

In some embodiments, a criteria may be defined as a flow rate that has decreased below a threshold for a sustained period of time, when the criteria is detected, an alert is sent to a caregiver or healthcare professional. Conveniently, this may enable timely detection of catheter blockages that can be detrimental to patient health especially when wound drains are used for clinical purposes and may assist clinicians in deciding when to remove a wound drain.

In some embodiments, a criteria may be defined as a concentration of amylase exceeds a threshold, for example, as detected by an amylase sensor as described herein, which may be indicative of a pancreatic leak. When the criteria is detected, an alert may be sent to a caregiver or healthcare professional.

Calibrator 1330 is configured to calibrate the settings of sensor device 710, for example, to standardize the signals being detected by sensors to the user's body.

In some embodiments, calibration may be performed by insertion of a calibration fluid through the catheter to sensor device 710.

Calibrator 1330 may operate on data collected while pumping different fluids into the catheter allowing the biosensors to calibrate, as described herein.

User profiler 1340 is configured to generate and update a profile of a user.

In some embodiments, signal data received from a sensor device may be aggregated and associated with a user, for example, over time, to develop a profile for that user. In some embodiments, user profile information, such as signal data associated with one or more users, may be applied to machine learning techniques to develop models for such signal data.

A user profile may include information about a user such as information related to a surgical procedure performed on the user and date and time of the surgical procedure, location of the surgical procedure, date and time of insertion of the sensor device, location of insertion of the sensor device, the user's age, height, weight, medical history, condition or illness (e.g., diabetic), current or past medication in use by the user, or other current or historical factors related to a user, surgery, or device details.

In some embodiments, a user profile includes a list of medications used by the user. This may be used to identify potential error sources caused by medication altering the threshold of one or more of the bio-signals being measured using sensors described herein.

In some embodiments, a user profile comprises the procedures that were performed on the patient. Such a list of procedures can be used to further analyze the potential list of complications that the user may suffer from given the risks for each procedure. Furthermore, such a list of procedures may used to identify the anatomy of biological fluids proximate to the procedure location.

In some embodiments, information related to a surgical procedure performed on the user includes a date and time of the surgical procedure. Furthermore the surgery date and time may be used to analyze the user condition given the full timeline of recovery for the user.

In some embodiments, a user profile data may be input by the user, a healthcare institution, or may be input by a healthcare professional, for example, a surgeon may input information related to a surgery that was performed and details regarding the sensor device (for example, operating parameters, the number and type of sensors, etc) being used following a surgery.

In some embodiments, a user profile may be automatically generated, for example, from health records indicating surgical details, or a user's electronic health record. These may be received from a computing device in communication with system 100.

In some embodiments, a user profile may include information identifying factors that are associated with a user condition determined from collected signal data.

In some embodiments, a user profile, including signal data, may be collected for one or more users of system 700 or instances of system 700.

User profile information, user conditions, and criteria may be stored in the user profile data store 1390.

A user profile may be updated, based at least in part on sensor data from sensors as described herein.

Signal data associated with a particular user may be used by predictor 1350 for further analysis of the signal data and for leak prediction, as discussed in further detail below.

Predictor 1350 is configured to execute data analysis and algorithms, such as machine learning techniques, to detect a leak and determine if a leak has occurred. In some embodiments, predictor 1350 may predict a future occurrence of a leak on the basis of data received from sensors. For example, a time at which the user condition occurs, and a length of time for which the user condition occurs.

In an example, a user condition dictated by sensor data that occurs for a temporary period of time, indicating, for example, a temporary spike, may be discarded as not indicating that particular condition, and instead an anomaly.

In some embodiments, the future occurrence of a complication, such as an anastomotic leak, can be predicted based at least in part on a time at which the user condition occurs, and a length of time for which the user condition occurs.

Machine learning algorithms may be applied to previously acquired signal data associated with a user condition. For example, pattern recognition may be performed on previously acquired signal data that is associated with a particular user condition. The machine leaning may generate a user condition classification model trained by the previously acquired signal data.

A leakage may be predicted by an analysis of a change in flow of fluid surrounding a sensor of the sensor device. For example, how fast a change in flow occurs may be indicative of how fast a leak is flowing.

In another example, a leak may be predicted on the basis of a build-up of lactate detected by a sensor.

In another example, a leak may be predicted on the basis of a depletion of oxygen detected by a sensor.

In another example, a leak may be predicted on the basis of a detected pH change, and may include an analysis of the why the pH has changed to differentiate between different causes or conditions for such a pH change. For example, predictor 1350 may differentiate between a pH change that is likely caused by a leak and a pH change that is caused by a medication being used by a user, which may be information generated or stored by user profiler 1340.

In some embodiments, a future occurrence of the anastomotic leak may be predicted based on a user's condition being above or below a predetermined threshold. Such a threshold may be, for example a pH value.

As described above, a secondary condition or second user condition may be determined from sensor data. The future occurrence of a leak may be predicted based at least in part on the second user condition. The second user condition may also indicate a risk factor or risk level of a leak condition.

In some embodiments, a Kalman filter may be used to predict if a leak has occurred.

Predictive analysis of biosignals may include a detection of certain physiological changes that typically occur before a leak develops, and the use of biomarkers that are related to such changes.

In some embodiments, predictor 1350 may determine confidence levels for whether a leak has occurred or not, based on a combined analysis of the user profile and processed biosignals. Weighted coefficients based on the user's profile and current condition may be used to contextualize algorithm inputs/outputs depending on the likelihood that a leak is developing. These weights may be dynamic over time and updated as the user's condition is updated. In an example, if a user had undergone bariatric surgery, higher weights may be applied to gastric leak detection algorithms, as compared to colorectal leak detection algorithms.

In some embodiments, predictive analysis of signals such as biosignals from a biosignal sensor, such as from sensor device 101 or inline monitoring device 1500, may include a diagnosis, for example, an identification of the nature of a leak or illness by examination of symptoms monitored by a sensor.

In some embodiments, a triage condition or risk level of a future occurrence prediction of a leak may be based on the signal data, the user condition, and the user profile. The data generated may include a triage condition or a risk level.

Machine learning algorithms may be applied to previously acquired signal data, user profile data, and user condition data. For example, pattern recognition may be performed on previously acquired signal data that is associated with a particular leak prediction.

Data associated with a future occurrence prediction of a leak may include a notification of the prediction.

In some embodiments, signal data may be collected to build a trend across a number of patients, and a cross-correlation technique may be used to identify the similarity between a patient's data and previous patients. And a match or correlation may indicate a risk factor.

Conveniently, it may be possible to make better predictions because there is a better fidelity of data acquired over time, for example, continuously as fluid flows through a sensor device, and in an example, in real-time or near real-time.

As will be appreciated, any or all of the hardware or software components described herein may be implemented and/or executed on a computing device such as an external computing device, for example, remote server 742, or a computing device on the sensor device.

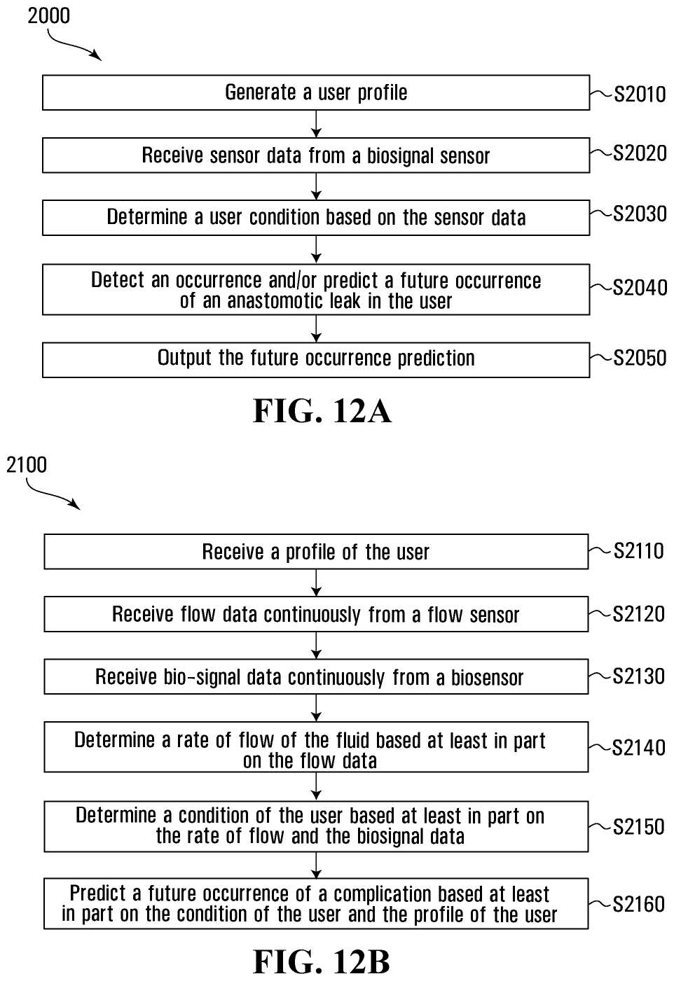

FIG. 12A illustrates a method 2000 of monitoring an anastomotic leak condition in a user. Blocks S2010 to S2050 may be performed by processor(s) 1210. The steps are provided for illustrative purposes. Variations of the steps, omission or substitution of various steps, or additional steps may be considered.

At block S2010, a user profile is generated by user profiler 1340. In some embodiments, a user profile may not be associated with use of monitoring for a leak.

At block S2020, sensor data is received from a sensor such as a biosignal sensor. The biosignal sensor may be disposed on a catheter as part of a sensor device 101 that is inserted in a user's body. In some embodiments, a sensor and/or sensor device 101 may be inserted in a part of the user's body, such as a cavity, that was subject to surgery that is being targeted for monitoring. In some embodiments, a sensor, for example, disposed on a sensor device, may be outside a user's body.

At block S2030, the processor operates to determine a user condition, based at least in part on the sensor data received.

At block S2040, an occurrence of an anastomotic leak in the user may be detected and/or a future occurrence of a leak predicted by predictor 1350. In an example, a future occurrence of an anastomotic leak may be predicted upon a user condition of a change in pH levels in a user's body.

In some embodiments, control flow may loop back to block S2020 such that a user's condition may be continuously updated (based on continuously-obtained sensor data) and used to predict future events.

At block S2050, the prediction of a future occurrence of an anastomotic leak is output. In an example, data may be output to display 736 of monitor 720, for viewing by the user.

In some embodiments, a leak may be detected on the basis of a change in pH values measured by one or more biosignal sensors.

In some embodiments, a leak may be detected on the basis of the presence of a fluid in the user's body.

In some embodiments, a leak may be detected by taking into account a user's profile, for example, the user's height, weight, age, and list of current medications in use.

In some embodiments, the occurrence of a leak in the future may be predicted based on the sensor data received. Prediction may be based on various conditions, or trends identified in biosignal data.

It should be understood that the blocks may be performed in a different sequence or in an interleaved or iterative manner.

FIG. 12B illustrates a method 2100 of monitoring a user. Blocks S2110 to S2160 may be performed by processor(s) 1210. The steps are provided for illustrative purposes. Variations of the steps, omission or substitution of various steps, or additional steps may be considered.

At block S2110, a profile of the user is received, including information related to a surgical procedure performed on a user.

At block S2120, flow data is continuously received from a flow sensor that is in fluid communication with fluid from a body of a user.

At block S2130, bio-signal data is continuously received from a biosensor that is in fluid communication with the fluid.

At block S2140, a rate of flow of the fluid is determined based at least in part on the flow data.

At block S2150, a condition of the user is determined based at least in part on the rate of flow and the bio-signal data.

At block S2160, a future occurrence of a complication is predicted, based at least in part on the condition of the user and the profile of the user.

It should be understood that the blocks may be performed in a different sequence or in an interleaved or iterative manner.

Example Application

An example application for systems and methods disclosed herein can be showcased by looking at a patient that is suffering from colorectal cancer, and the tumor needs to be removed. The surgeon may decide to perform an anastomosis after removing the tumor from the body. The surgeon may then place the catheter with biosensors in one of the paracolic gutters if they think it is the most likely region to collect fluid from a leak. The catheter system that they utilize may be equipped with fluid, pH and lactate sensors. Once the catheter has been placed, the surgeon may also choose to use absorbable sutures to keep the catheter held in place. The catheter may then be connected to a monitor placed outside the body. The catheter may also connect to a balloon which would apply negative pressure to drain fluid from the peritoneum. The monitor would then confirm that a connection has been established with the biosensors, informing the user and the surgeon that the patient condition appears to be normal. The patient may then be kept in the hospital overnight and then discharged the second day. The patient may be discharged with the monitor and the catheter. Three days following the surgery and after the patient has been discharged the biosensors may detect clinically relevant pH change and simultaneous increases in lactate concentration. The monitor may then signal to the patient to seek medical attention, or the system may delay the signal to wait for more significant changes. The data may be relayed wirelessly to the medical facility, at which point a specialist may look at the data and also make a decision of whether to have the patient come to the medical facility or not. The system may then detect a significant pH change and a relatively high flow of fluid in the abdominal cavity. The monitor may then alert the patient informing them that a leak has been detected and that they need to seek medical intervention immediately. The medical facility may also be notified. Once the patient is at the hospital, the medical facility may look at the data obtained from the biosensors and make a clinical decision to support the patient before the complication grows. A surgeon may also decide to take corrective medical action, including but not limited to re-operation on the patient. The system may be utilized again after the corrective action is done.

Conveniently, a sensor device as described herein may be removable from a user or patient in an outpatient setting, for example, in a user's home by a nurse, and without the need for a user to undergo an additional surgical procedure. In an example, this may occur ten to twenty days following a surgery.

In some embodiments, integrating sensors with catheters (and specifically, drains) may allow a sensor device to be removed in an outpatient setting.

### Experimental Data

In experimental work to date related to detecting anastomotic leaks post-surgery recording preliminary animal study data using a pig model, there is evidence of a drop in pH due to leakage of highly acidic gastric juice.

For preliminary animal studies, surgeries were performed on each pig at the stomach. Two pH biosensors were implanted near the surgical site then a leakage induced.

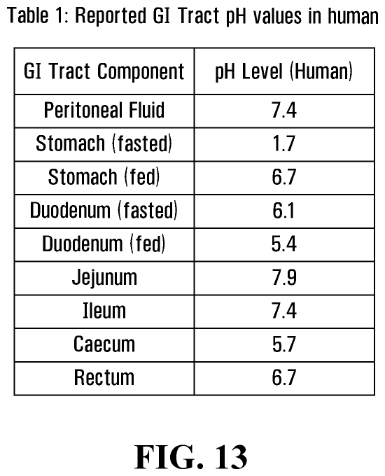

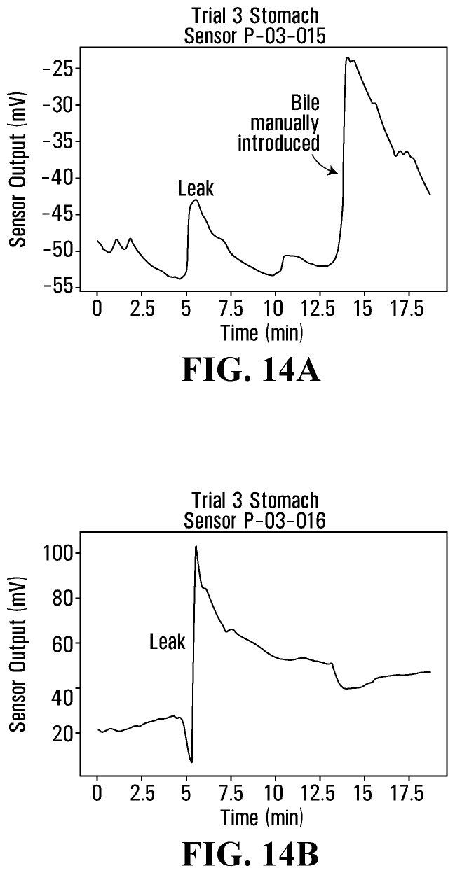

Table 1, as shown in FIG. 13 illustrates reported GI tract pH values in a human.

For a first study, two sensors were connected to Digilent Analog Discovery 1, and the remaining four sensors were connected to two Digilent Analog Discovery 2 devices. Data for all the study sensors was recorded using Digilent Waveforms software.

FIGS. 14B and 14B illustrates stomach sensor data captured during the study (“Trial 3 Stomach”). Graphs in FIGS. 14B and 14B show data for sensors P-03-015 and P-03-016 respectively. Sensor P-03-016 was placed closer to the leakage site and a larger spike is observed when leak was introduced around 5 min. Furthermore, bile was extracted at the end of the study and manually added on top of sensor P-03-015. As shown, a big spike was observed.

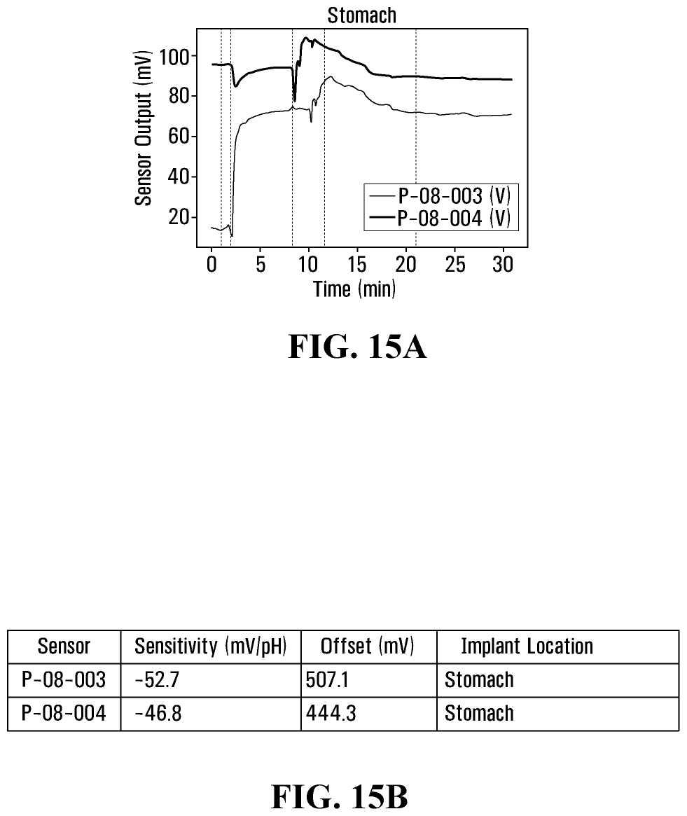

FIG. 15A illustrates filtered sensor data captured during a second study. The graphs show sensors implanted next to the stomach. Each graph represents the pH sensor output in mV per location. The vertical dotted lines represent the different surgical events during the trial. FIG. 15B illustrates details of the further study of FIG. 15B, including sensitivity offset and location for each sensor. At 1 minute, a control occurred and a surgeon pretended to induce a leak, but did not actually. At 2 minutes, a gastric leak event occurred. At 8 minutes, an intestinal leak event occurred. At 12 minutes, a rectal leak event occurred. At 21 minutes an intestinal leak event occurred.

Figures showing real-time sensor measurements are filtered to remove anomalies (e.g., due to connector issues), reduce noise, and trimmed in time to only show relevant intervals.

Although pig models were used in these preliminary studies, pigs possess a GI tract that is very similar in structure and function to that of a human's. A pig's digestive system organs operate almost identically to a human's; however, they are much greater in size. The GI tract pH levels are estimated to be similar in humans and pigs.

All data measured in the first study was recorded on Analog Discovery devices and no multimeters were used. The first study graphs (FIGS. 14A, 14B) show the real time progress of all the sensors and leakage was induced around 5-7.5 min. Sensor P-03-016 measured an estimated decrease of 1.9 pH, while sensor P-03-015 only measured an estimated decreased of 0.1 pH. However, it was observed that the amount of gastric juice leaking in this study was less than that of previous studies. Additionally, sensor P-03-015 was implanted farther away from the leak and likely did not interface with the leaked fluid directly, hence the slight response.

In conclusion, sensors implanted near the stomach demonstrated significant drops in pH due to the leakage of highly acidic gastric juice.

The results validate the use of a sensor device in the GI tract near the surgical sites to detect anastomotic leaks post-surgery. The trend of pH changes can be observed in all three locations.

In some embodiments, sensors may be characterized and calibrated to simulate the implant environment (e.g., sensors may be characterized at 37 degrees C.).

In some embodiments, techniques may be implemented to reduce mismatch between sensor offsets and reduce temporal offset drift. This may entail the use of a different material as a PR.

In some embodiments, use of a custom acquisition setup may reduce noise, isolate sensors from powerlines for safety, attenuate temporal drifts, reduce sensor leakage currents, and reduce sensor cross-talk to allow for more accurate estimation of pH changes.

Of course, the above described embodiments are intended to be illustrative only and in no way limiting. It will be apparent to those skilled in the art that various modifications and variations may be made in the materials, devices and methods disclosed herein. It will be understood that elements of embodiments are not necessarily mutually exclusive, and many embodiments can suitably be combined with other embodiments. For example, sensor devices may be manufactured using certain various combinations of components such as sensors, with certain various methods and in combination with supporting substrates of different materials, shapes and configurations.

The examples described above and illustrated are intended to be exemplary only. The description shall be understood to encompass all equivalents.

The described embodiments are susceptible to many modifications of form, arrangement of parts, details and order of operation. The disclosure is intended to encompass all such modification within its scope, as defined by the claims.

1-25. (canceled)

26. A monitoring device comprising:
an input port, attachable to a catheter for insertion in a body of a user, the input port in fluid communication with the catheter for receiving fluid from the body of the user;
an output port in fluid communication with a fluid reservoir;
a fluid channel defining fluid communication between the input port and the output port; and
a biosensor, in communication with a computing device, for continuously measuring bio-signal data of the fluid in the fluid channel.

27. The monitoring device of claim 26, wherein the output port is generally parallel to the input port.

28. The monitoring device of claim 26, wherein the computing device is for determining a condition of the user based at least in part on the bio-signal data.

29. The monitoring device of claim 26, wherein the biosensor includes an impedance sensor for detecting an impedance or conductivity of the fluid in the fluid channel.

30. The monitoring device of claim 26, wherein the biosensor includes a pH sensor for detecting a pH level in the fluid in the fluid channel.

31. The monitoring device of claim 26, wherein the biosensor includes at least one of a lactate sensor, an amylase sensor, a urea sensor, or a creatinine sensor.

32. The monitoring device of claim 26, wherein the biosensor is a flow sensor for continuously determining a flow rate of the fluid in the fluid channel over time.

33. The monitoring device of claim 26, wherein the biosensor is a light-based sensor including a light transmitter and a light receiver for detecting transmission of light through the fluid in the fluid channel.

34. The monitoring device of claim 33, wherein the light-based sensor is configured to detect a colour of the fluid based at least in part on a detected wavelength.

35. The monitoring device of claim 26, further comprising a temperature sensor for detecting a temperature of the fluid in the fluid channel.

36. The monitoring device of claim 26, wherein the biosensor is disposed on a substrate in fluid communication with the fluid channel.

37. The monitoring device of claim 26, wherein the biosensor is disposed along a length of the fluid channel.

38. A computer-implemented method for monitoring a user, the method comprising:
receiving bio-signal data continuously from a biosensor in fluid communication with the fluid;
determining a condition of the user based at least in part on the bio-signal data; and
predicting a future occurrence of a complication based at least in part on the condition of the user.

39. The method of claim 38, further comprising:
receiving a profile of the user, the profile of the user including at least one of; information related to a surgical procedure performed on the user, date and time of the surgical procedure, location of the surgical procedure, date and time of insertion of the biosensor, location of insertion of the biosensor, the user's age, height, weight, medical history, condition or illness, current or past medication in use by the user, current or historical factors related to a user, or biosensor details,
wherein the determined condition of the user or future occurrence of the complication is predicted based at least in part on the profile of the user.

40. The method of claim 39, further comprising updating the profile of the user based at least in part on the bio-signal data.

41. The method of claim 38, further comprising:
receiving flow data continuously from a flow sensor in fluid communication with fluid from a body of a user; and
determining, based at least in part on the flow data, a rate of flow of the fluid,
wherein the condition of the user is determined based at least in part on the rate of flow.

42. The method of claim 41, further comprising determining a rate of change in the rate of flow of the fluid over time and a rate of change in bio-signal data over time, and the predicting the future occurrence is based at least in part on the rate of change in the rate of flow and the rate of change in bio-signal data.

43. The method of claim 41, wherein the flow data is received in near real-time.

44. The method of claim 38, wherein the bio-signal data is received in near real-time.

45. The method of claim 38, further comprising receiving light data associated with transmission of light through the fluid from a light-based sensor in fluid communication with the fluid.

46. The method of claim 45, further comprising determining a color of the fluid based at least in part on the light data.

47. The method of claim 38, further comprising receiving temperature data of the fluid from a temperature sensor in fluid communication with the fluid.

48. The method of claim 47, further comprising modulating the bio-signal data based at least in part on the temperature data.

49. The method of claim 38, further comprising determining a risk factor of the user based on a cross-correlation with a trend of bio-signal data of other users.

50. The method of claim 38, wherein the condition of the user is based at least in part on determining whether the bio-signal data is within bounds of a threshold.

51. A system for monitoring a user, comprising:
a processor;
a memory in communication with the processor, the memory storing instructions that, when executed by the processor cause the processor to perform the method of claim 38.
